(19) United States
(12) Patent Application Publication
(10) Pub. No.: US 2005/0090732 A1
(43) Pub. Date: Apr. 28, 2005

(54) Therapy via targeted delivery of nanoscale particles

(75) Inventors: Robert Ivkov, Marblehead, MA (US); Wolfgang Daum, Groton, MA (US); Allan Foreman, Epping, NH (US); Douglas Gwost, Shoreview, MN (US)

Correspondence Address: PEPPER HAMILTON LLP, ONE MELLON CENTER, 50TH FLOOR, 500 GRANT STREET, PITTSBURGH, PA, 15219, US

(73) Assignee: Triton Biosystems, Inc., Chelmsford, MA (US)

(21) Appl. No.: 10/696,399

(22) Filed: Oct. 28, 2003

(51) Int. Cl. A61B 5/055

(52) U.S. Cl. 600/411; 324/318

(57) ABSTRACT

Disclosed are compositions, systems and methods for treating a subject's body, body part, tissue, body fluid cells, pathogens, or other undesirable matter involving the administration of a targeted thermotherapy that comprises a bioprobe (energy susceptive materials that are attached to a target-specific ligand). Such targeted therapy methods can be combined with at least one other therapy technique. Other therapies include hyperthermia, direct antibody therapy, radiation, chemo- or pharmaceutical therapy, photodynamic therapy, surgical or interventional therapy, bone marrow or stem cell transplantation, and medical imaging, such as MRI, PET, SPECT, and bioimpedance. The disclosed therapies may be useful in the treatment of a variety of indications, including but not limited to, cancer of any type, such as bone marrow, lung, vascular, neuro, colon, ovarian, breast and prostate cancer, epitheleoid sarcomas, AIDS, adverse angiogenesis, restenosis, amyloidosis, tuberculosis, cardiovascular plaque, vascular plaque, obesity, malaria, and illnesses due to viruses, such as HIV.

### TECHNICAL FIELD

The present invention relates generally to targeted therapeutic compositions, systems and methods. Specifically, the invention pertains to compositions, systems and methods pertaining to devascularization using thermotherapy. In addition, the invention pertains to a combination of a thermotherapy method with at least one other treatment, where the targeted thermotherapy comprises the administration of an energy susceptive material, which is attached to a target-specific ligand, to a subject's body, body part, tissue, or body fluid, and the administration of energy from an energy source, so as to destroy or inactivate the target.

### BACKGROUND

The time between the onset of disease in a patient and the conclusion of a successful course of therapy is often unacceptably long. Many diseases remain asymptomatic and evade detection while progressing to advanced, and often terminal, stages. In addition, this period may be marked by significant psychological and physical trauma for the patient due to the unpleasant side effects of even correctly prescribed treatments. Even diseases that are detected early may be most effectively treated only by therapies that disrupt the normal functions of healthy tissue or have other unwanted side effects.

One such disease is cancer. Despite considerable research effort and some success, cancer is still the second leading cause of death in the United States, claiming more than 500,000 lives each year according to American Cancer Society estimates. Traditional treatments are invasive and/or are attended by harmful side effects (e.g., toxicity to healthy cells), often making for a traumatic course of therapy with only modest success. Early detection, a result of better diagnostic practices and technology, has improved the prognosis for many patients. However, the suffering that many patients must endure makes for a more stressful course of therapy and may complicate patient compliance with prescribed therapies. Further, some cancers defy currently available treatment options, despite improvements in disease detection. Of the many forms of cancer that still pose a medical challenge, prostate, breast, lung, and liver claim the vast majority of lives each year. Colorectal cancer, ovarian cancer, gastric cancer, leukemia, lymphoma, melanoma, and their metastases may also be life threatening.

Conventional treatments for breast cancer, for example, typically include surgery followed by radiation and/or chemotherapy. These techniques are not always effective, and even if effective, they suffer from certain deficiencies. Surgical procedures range from removal of only the tumor (lumpectomy) to complete removal of the breast. In early stage cancer, complete removal of the breast may provide an assurance against recurrence, but is disfiguring and requires the patient to make a very difficult choice. Lumpectomy is less disfiguring, but can be associated with a greater risk of cancer recurrence. Radiation therapy and chemotherapy are arduous and are not completely effective against recurrence.

Treatment of pathogen-based diseases is also not without complications. Patients presenting symptoms of systemic infection are often mistakenly treated with broad-spectrum antibiotics as a first step. This course of action is completely ineffective when the invading organism is viral. Even if a bacterium (e.g., E. coli) is the culprit, the antibiotic therapy eliminates not only the offending bacteria, but also benign intestinal flora in the gut that are necessary for proper digestion of food. Hence, patients treated in this manner often experience gastrointestinal distress until the benign bacteria can repopulate. In other instances, antibiotic-resistant bacteria may not respond to antibiotic treatment. Therapies for viral diseases often target only the invading viruses themselves. However, the cells that the viruses have invaded and “hijacked” for use in making additional copies of the virus remain viable. Hence, progression of the disease is delayed, rather than halted.

For these reasons, it is desirable to provide improved and alternative techniques for treating disease. Such techniques should be less invasive and traumatic to the patient than the present techniques, and should only be effective locally at targeted sites, such as diseased tissue, pathogens, or other undesirable matter in the body. Preferably, the techniques should be capable of being performed in a single or very few treatment sessions (minimizing patient non-compliance), with minimal toxicity to the patient. In addition, the undesirable matter should be targeted by the treatment without requiring significant operator skill and input.

Immunotherapy is a rapidly expanding type of therapy used for treating a variety of human diseases including cancer, for example. The FDA has approved a number of antibody-based cancer therapeutics. The ability to engineer antibodies, antibody fragments, and peptides with altered properties (e.g., antigen binding affinity, molecular architecture, specificity, valence, etc.) has enhanced their use in therapies. Cancer immunotherapeutics have made use of advances in the chimerization and humanization of murine antibodies to reduce immunogenic responses in humans. High affinity human antibodies have also been obtained from transgenic animals that contain many human immunoglobulin genes. In addition, phage display technology, ribosome display, and DNA shuffling have allowed for the discovery of antibody fragments and peptides with high affinity and low immunogenicity for use as targeting ligands. All of these advances have made it possible to design an immunotherapy that has a desired antigen binding affinity and specificity, and minimal immune response.

The field of cancer immunotherapy makes use of markers that are over-expressed by cancer cells (relative to normal cells) or expressed only by cancer cells. The identification of such markers is ongoing and the choice of a ligand/marker combination is critical to the success of any immunotherapy. Immunotherapeutics fall into at least three classes: (1) deployment of antibodies that, themselves, target growth receptors, disrupt cytokine pathways, or induce complement or antibody-dependent cytotoxicity; (2) direct arming of antibodies with a toxin, a radionuclide, or a cytokine; (3) indirect arming of antibodies by attaching them to immunoliposomes used to deliver a toxin or by attaching them to an immunological cell effector (bispecific antibodies). Although armed antibodies have shown potent tumor activity in clinical trials, they have also exhibited unacceptably high levels of toxicity to patients.

The disadvantage of therapies that rely on delivery of immunotoxins or radionuclides (i.e., direct and indirect arming) has been that, once administered to the patient, these agents are active at all times. These therapies often cause damage to non-tumor cells and present toxicity issues and delivery challenges. For example, cancer cells commonly shed surface-expressed antigens (targeted by immunotherapeutics) into the blood stream. Immune complexes can be formed between the immunotherapeutic and the shed antigen. As a result, many antibody-based therapies are diluted due to the interaction of the antibody with these shed antigens rather than interacting with the cancer cells, and thereby reducing the true delivered dose. Thus, a “therapy-on-demand” approach that minimizes adverse side effects and improves efficacy would be preferable.

With thermotherapy, temperatures in a range from about 40° C. to about 46° C. (hyperthermia) can cause irreversible damage to disease cells. However, healthy cells are capable of surviving exposure to temperatures up to about 46.5° C. Elevating the temperature of individual cells in diseased tissue to a lethal level (cellular thermotherapy) may provide a superior treatment option. Pathogens implicated in disease and other undesirable matter in the body can also be destroyed via exposure to locally high temperatures.

Hyperthermia may hold promise as a treatment for cancer and other diseases because it induces instantaneous necrosis (typically called “thermo-ablation”) and/or a heat-shock response in cells (classical hyperthermia), leading to cell death via a series of biochemical changes within the cell. State-of-the-art systems that employ microwave or radio frequency (RF) hyperthermia, such as annular phased array systems (APAS), attempt to tune energy for regional heating of deep-seated tumors. Such techniques are limited by the heterogeneities of tissue and to highly perfused tissue. This leads to the as-yet-unsolved problems of “hot spot” phenomena in untargeted tissue with concomitant underdosage in the desired areas. These factors make selective heating of specific regions with such systems very difficult.

Another strategy that utilizes RF hyperthermia requires surgical implantation of microwave or RF based antennae or self-regulating thermal seeds. In addition to its invasiveness, this approach provides few (if any) options for treatment of metastases because it requires knowledge of the precise location of the primary tumor. The seed implantation strategy is thus incapable of targeting undetected individual cancer cells or cell clusters not immediately adjacent to the primary tumor site. Clinical success of this strategy is hampered by problems with the targeted generation of heat at the desired tumor tissues.

### SUMMARY OF THE INVENTION

Hyperthermia for treatment of disease using energy sources exterior to the body has been recognized for several decades. However, a major problem has been the inability to selectively deliver a lethal dose of heat to the cells or pathogens of interest.

In view of the above, there is a need for a thermotherapeutic method for treating diseased tissue, pathogens, or other undesirable matter that incorporates selective delivery of energy to a target within a subject's body, especially for devascularization. There is also a need for combined therapy methods for treating diseased tissue, pathogens, or other undesirable matter that include targeted thermotherapy.

It is, therefore, an aspect of the present invention to provide a treatment method that involves the administration of energy susceptive materials that are attached to a target-specific ligand, to a subject's body, body part, tissue, or body fluid, and the administration of an energy source to inhibit or destroy the vascularity of the tumor (devascularization).

It is also an aspect of the present invention to provide a treatment method that involves the administration of energy susceptive materials that are attached to a target-specific ligand, to a subject's body, body part, tissue, or body fluid, and the administration of an energy source to destroy, rupture, or inactivate the target (targeted thermotherapy) that can be utilized in combination with other treatments.

It is another aspect of the present invention to administer the energy to a selected cell or tissue, to a subject's entire body, or extracorporeally to the subject's body, organ or body fluid.

The present invention pertains to thermotherapy methods that comprise the administration of a bioprobe (energy susceptive particles that are attached to a target-specific ligand) to a subject, and administration of an energy source to the bioprobe, after a prescribed period of time for the bioprobe to locate and attach to a markered target, so as to destroy or inactivate the target or inhibit or destroy the vascularity of the tumor. The present invention also pertains to thermotherapy using the combination of targeted thermotherapy and at least one other treatment. The energy may be administered directly into the subject's body, body part, tissue, or body fluid (such as blood, blood plasma, blood serum, or bone marrow), or extracorporeally to the subject's body, organ or body fluid.

The combination therapy methods of the present invention involve the thermotherapy methods and devices disclosed in commonly owned U.S. Patent Applications US2003/0032995, US2003/0028071, 10/360,578, and 10/360,561 (each of which is incorporated herein by reference) with at least one other treatment. The other treatments include, for instance, direct antibody therapy; hyperthermia heating which includes eddy current, RF, and microwave radiation, direct AC or DC currents, thermal seeds, thermal bath, non-targeted particle heating, and heating by ionizing radiation; radiation therapy which includes external beam radioimmuno therapy, internal radiotherapy, targeted isotopes, and radiation activated therapy; chemotherapy and pharmaceutical therapy, systemic or local delivery, local implanted delivery, antibody targeted, and light activated pharmaceuticals; photodynamic therapy (PDT); surgery and interventional techniques; bone marrow and stem cell transplantation; and medical imaging.

The invention pertains to a targeted thermotherapy system for treating disease material in a patient. The system includes a bioprobe or a bioprobe system comprising a susceptor, an alternating magnetic field (AMF) inducing inductor that produces an AMF to energize the susceptor; and a generator coupled to the inductor to provide power to the AMF inducing inductor.

The invention also pertains to therapeutic method for treating the body, body part, tissue, cell, or body fluid of a subject. The method comprises administering targeted thermotherapy to a target by supplying a bioprobe to the target and exposing the bioprobe to an alternating magnetic field (AMF), and administering at least one other therapy to the target. The at least one other therapy is administered prior to, during, after the targeted thermotherapy administration, or a combination thereof.

The invention also pertains to a therapeutic method comprising administering targeted thermotherapy to a body, body part, or tissue of a subject containing a tumor, by supplying a bioprobe to the body, body part or tissue and exposing the bioprobe to an alternating magnetic field (AMF), and destroying or inhibiting the vascularity of the body, body part or tissue in response to exposure to the AMF.

Further, the invention pertains to a therapeutic method for treating the body, body part, tissue, cell, or body fluid of a subject. The method comprises medically imaging the body, body part, tissue, cell or body fluid; and administering targeted thermotherapy by introducing a bioprobe to the body, body part, tissue, cell or body fluid of the subject and exposing the bioprobe to an alternating magnetic field (AMF). Administering the targeted thermotherapy occurs prior to, during, or after the medical imaging, or a combination thereof.

The invention also pertains to a magnetic material composition. The composition comprises a particle having magnetic properties and forming a single magnetic domain; a biocompatible coating material for the particle; and a ligand selective to at least one disease material marker associated with disease material. The ligand can be i) bound to an uncoated portion of the particle, ii) bound to a coated portion of the particle, iii) bound to the particle and partially covered by the coating or iv) intercalated into the coating.

In addition, the invention also relates to a magnetic material composition. The composition comprises a bioprobe, the bioprobe comprising a particle having magnetic properties associated with a first therapy, and a ligand selective to at least one disease material marker associated with a disease material; the ligand being associated with the particle The composition also comprises an agent associated with a second therapy, the agent being associated with the bioprobe.

The above summary of the present invention is not intended to describe each illustrated embodiment or every implementation of the present invention. The figures and the detailed description that follow particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 schematically illustrates a thermotherapy treatment system, according to an embodiment of the present invention;

FIG. 2 schematically illustrates a thermotherapy treatment, according to an embodiment of the present invention;

FIG. 3 schematically illustrates a bioprobe configuration, according to an embodiment of the present invention;

FIG. 4 schematically illustrates a disease specific targeting ligand component of a bioprobe, according to an embodiment of the present invention;

FIG. 5 schematically illustrates disease specific bioprobes bound to a disease cell surface, according to an embodiment of the present invention;

FIG. 6 schematically illustrates a circuit for producing a thermotherapeutic alternating magnetic field, according to an embodiment of the present invention;

FIG. 7 schematically illustrates a means for generating AMF, according to an embodiment of the present invention;

FIG. 8 illustrates a cross sectional view of an inductor configuration, according to an embodiment of the present invention;

FIG. 9 is a block diagram illustrating an embodiment of the targeted therapeutic system, according to an embodiment of the present invention;

FIGS. 10a and 10b schematically illustrate two types of electrical field shielding for the inductor, according to an embodiment of the present invention;

FIG. 11 schematically illustrates a bioprobe configuration comprising a radio tag, according to an embodiment of the present invention; and

FIG. 12 schematically illustrates a bioprobe configuration comprising a chemotherapeutic agent, according to an embodiment of the present invention;

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention as defined by the appended claims.

### DETAILED DESCRIPTION OF EXEMPLARY EMBODIMENTS

The present invention pertains to devices for treating diseased, disease-causing, or undesirable tissue or material, for use with magnetic material compositions and methods for treating or removing the tissue or material utilizing such devices. The therapeutic methods disclosed herein include the targeted delivery of nanometer sized magnetic particles to the desired or target material.

1. Definitions

The term “bioprobe”, as used herein, refers to a composition comprising a susceptor and at least one ligand. The ligand acts to guide the bioprobe to a target.

The term “disease material”, as used herein, refers to diseased, disease-causing, or undesirable material in the body or body part of a subject.

The term “susceptor”, as used herein, refers to a particle (optionally comprising a coating) of a material that, when exposed to an energy source, either heats or physically moves. Similarly, the term “magnetic susceptor” refers to such particles wherein the energy source to which the particles respond is an alternating magnetic field (AMF).

The term “ligand”, as used herein, refers to a molecule or compound that attaches to a susceptor (or a coating on the susceptor) and targets and attaches to a biological marker. A monoclonal antibody specific for Her-2 (an epidermal growth factor receptor protein) is an exemplary ligand.

The term “target”, as used herein, refers to the matter for which deactivation, rupture, disruption or destruction is desired, such as a diseased cell, a pathogen, or other undesirable matter. A marker may be attached to the target. Breast cancer cells are exemplary targets.

The term “marker”, as used herein, refers to an antigen or other substance to which the bioprobe ligand is specific. Her-2 protein is an exemplary marker.

The term “bioprobe system”, as used herein, refers to a bioprobe specific to a target that is optionally identified via a marker.

The term “indication”, as used herein, refers to a medical condition, such as a disease. Breast cancer is an exemplary indication.

The term “energy source”, as used herein, refers to a device that is capable of delivering energy to the bioprobe's susceptor.

The term “AMF” (an abbreviation for alternating magnetic field), as used herein, refers to a magnetic field that changes the direction of its field vector periodically, for example in a manner that is sinusoidal, triangular, or rectangular. The AMF may also be added to a static magnetic field, such that only the AMF component of the resulting magnetic field vector changes direction. It will be appreciated that an alternating magnetic field is accompanied by an alternating electric field and is electromagnetic in nature.

The term “RF” (an abbreviation for radio frequency), as used herein, refers to a radio frequency in the range from about 0.1 Hz to about 900 MHz.

The term “duty cycle”, as used herein, refers to the ratio of the time that the energy source is on to the total time that the energy source is on and off in one on-off cycle.

The term “hyperthermia”, as used herein, refers to heating of tissue to temperatures between 40° C. and 45° C.

The term “light”, as used herein, refers to ultra violet (UV) light, infrared (IR) light, or light at any other wavelength, or to light in laser form.

The terms “targeted thermotherapeutic system”, “therapy system”, “targeted therapy”, “thermotherapy” and “therapy source”, as used herein, refer to the methods and devices that involve the targeted delivery of bioprobes for the treatment of an indication, including those disclosed in U.S. Patent Applications US2003/0032995, US2003/0028071, 10/360,578, and 10/360,561.

It is to be understood that the singular forms of “a”, “an”, and “the”, as used herein and in the appended claims, include plural reference unless the context clearly dictates otherwise.

2. The Targeted Thermotherapeutic System

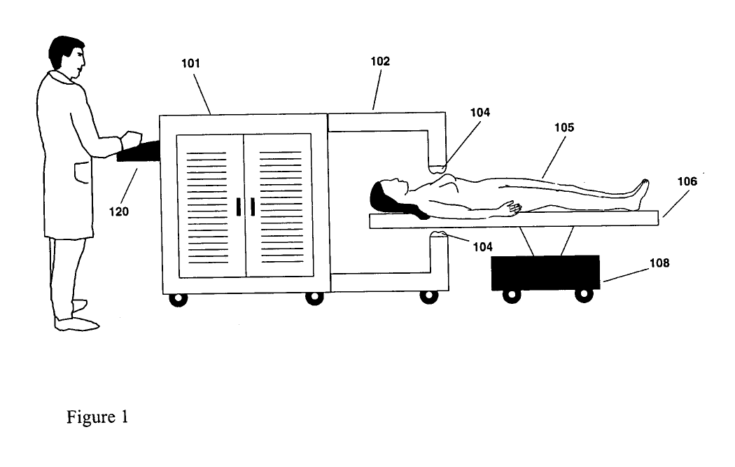

The targeted thermotherapy system, an embodiment of which is illustrated in FIG. 1, includes an energy source, e.g., an alternating magnetic field (AMF) generator 101 for producing an alternating magnetic field that may be guided to a specific location within a patient 105 by a magnetic circuit 102. The therapeutic methods of the present invention may be performed following a determination of the presence of disease material in one or more areas of the patient. For example, the disease material may be any one or combination of cancers and cancerous tissue, a pathogenic infection (e.g., viral, bacterial or multicellular parasitic), toxin, or any pathogen-like material (e.g., a prion). The manner of making the diagnosis does not form part of the invention and may be performed using any standard method. However, the present invention, or aspects thereof, may be amenable to a diagnostic function alone or in conjunction with another method or apparatus. Such a diagnostic function may be performed by using a suitable technology or technique to interrogate the magnetic properties of the bioprobes, and thus evaluate their concentration and location within the patient. The location and concentration of bioprobes may each be determined using an existing technique, such as magnetic resonance imaging, or another diagnostic technique can be established and performed using a suitable magnetometer, such as a Superconducting Quantum Interference Device (SQUID). Information obtained from this interrogation may be used to define the parameters of treatment, i.e. the location, duration, and intensity of the alternating magnetic field. The patient may be positioned on an X-Y horizontal and vertical axis positioning bed 106. Bed 106 may be both horizontally and vertically positionable using a bed controller 108. In one embodiment of the present invention, the AMF generator produces an AMF in magnetic circuit 102 that exits the magnetic circuit at one pole face 104, passing through the air gap and the desired treatment area of the patient, and reenters the circuit through the opposing pole face 104, thus completing the circuit. An operator or medical technician is able to both control and monitor the AMF characteristics and bed positioning via a control panel 120.

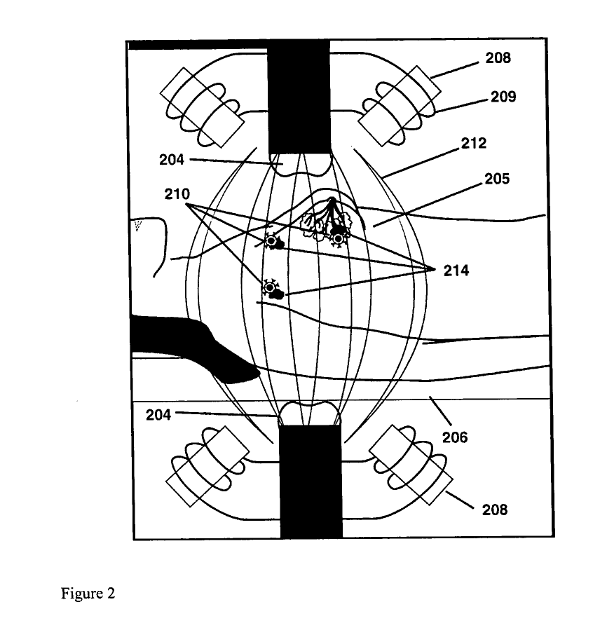

FIG. 2 illustrates a treatment of a patient with a device for treating disease material according to an embodiment of the present invention. The area of the patient to be treated 205 is localized in the region between the magnetic poles 204 using a positionable bed 206. This region may be any location of the patient including the chest, abdomen, head, neck, back, legs, arms, or any location of the skin. An AMF may be applied to treatment area 205 of the patient. The magnetic field, shown as lines of magnetic flux 212, interacts with both healthy and disease material in the localized area. Bioprobes 210, containing at least one appropriate ligand selective to the particular type of disease material, are bound to a disease material 214, or at least in the vicinity of the disease material. In the illustrated case, bioprobes 210 are selective to breast cancer. Bioprobes 210 become excited by the applied AMF and are inductively heated to a temperature sufficient to kill or render ineffective the disease material. For example, heat generated in the bioprobes 210 may pass to the cells, thereby causing the cells to die.

Furthermore, poles 204 may be formed from pieces whose gap is adjustable, so as to permit other parts of the body to be treated. It is advantageous to set the gap between poles 204 to be sufficiently large to permit the part of the body containing the disease material to enter the gap, but not be so large as to reduce the magnetic field strength. Also shown are secondary coils 208 and optional cores 209. Any number of these secondary coils and optional cores may be added to modify the distribution of magnetic flux produced by the primary coils 208′ and the core between the poles 204. Secondary coils 208 may be wired in series or in parallel with the primary coils 208′, or they can be driven by separate AMF generators. The phase, pulse width and amplitude of the AMF generated by these coils may be adjusted to maximize the field strength in the gap, minimize the field strength in areas which may be sensitive to AMF, or to uniformly distribute the magnetic field strength in a desired manner.

The targeted thermotherapy system may be used to administer a treatment to a subject intracorporeally (within the patient), extracorporeally (external to the patient), or a combination thereof. In extracorporeal therapy, bioprobes may be used to lyse, denature, or otherwise destroy the desired targets by circulating the blood outside of the body, exposing it to AMF, and returning it to the body. When the bioprobe/target complexes are carried primarily in the blood serum or plasma, the blood serum or plasma may be extracorporeally separated from the other blood components, exposed to AMF to destroy the target, and recombined with the other blood components before returning the blood to the body. The bioprobes may also be contained in a vessel or column through which the blood circulating outside of the body or the blood serum or plasma flows. The vessel or column may be exposed to AMF to destroy the targets before the blood is returned to the body. When the fluid is treated extracorporeally, the bioprobes may be introduced to the fluid after it has been extracted from the patient, or before extraction.

2.1 The Bioprobe of the Targeted Thermotherapy System

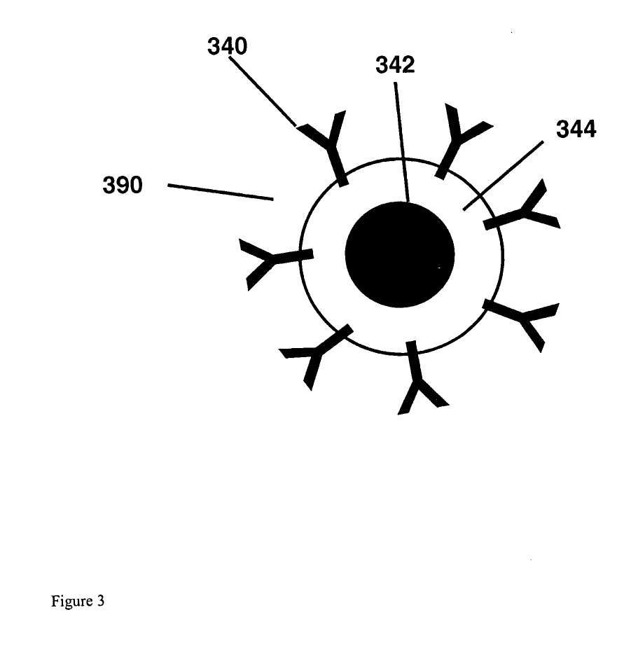

FIG. 3 discloses a bioprobe configuration according to an embodiment of the present invention. A bioprobe 390 comprises a magnetic energy susceptive particle 342. The magnetic particle 342, also referred to as a susceptor, may include a coating 344. Coating 344 may fully or partially coat susceptor 342. At least one targeting ligand 340, such as, but not limited to, an antibody, may be located on an exterior portion of bioprobe 390. The targeting ligand 340 may be selected to seek out and attach to a target, such as a particular type of cell or disease matter. Heat is generated in the susceptor 342 when susceptor 342 is exposed to an energy source, such as AMF. Coating 344 may enhance the heating properties of bioprobe 390, particularly if coating 344 has a high viscosity, for example, is a polymeric material.

In a general sense, this heat represents an energy loss as the magnetic properties of the material are forced to oscillate in response to the applied alternating magnetic field. The amount of heat generated per cycle of magnetic field and the mechanism responsible for the energy loss depend on the specific characteristics of both the susceptor 342 and the magnetic field. Susceptor 342 heats to a unique temperature, known as the Curie temperature, when subjected to an AMF. The Curie temperature is the temperature of the reversible ferromagnetic to paramagnetic transition of the magnetic material. Below this temperature, the magnetic material heats in an applied AMF. However, above the Curie temperature, the magnetic material becomes paramagnetic and its magnetic domains become unresponsive to the AMF. Thus, the material does not generate heat when exposed to the AMF above the Curie temperature. As the material cools to a temperature below the Curie temperature, it recovers its magnetic properties and resumes heating, as long as the AMF remains present. This cycle may be repeated continuously during exposure to the AMF. Thus, magnetic materials are able to self-regulate the temperature of heating. The temperature to which susceptor 342 heats is dependent upon, inter alia, the magnetic properties of the material, characteristics of the magnetic field, and the cooling capacity of the target site. Selection of the magnetic material and AMF characteristics may be tailored to optimize treatment efficacy of a particular tissue or target type. In an embodiment of the present invention, the magnetic material may be selected to possess a Curie temperature between about 40° C. and about 150° C.

Many aspects of susceptor 342, such as material composition, size, and shape, directly affect heating properties. Many of these characteristics may be designed simultaneously to tailor the heating properties for a particular set of conditions found within a tissue type. For example, for susceptor 342, the most desirable size range depends upon the particular application and on the material(s) comprising susceptor 342.

The size of susceptor 342 determines the total size of bioprobe 390. Bioprobes 390 that are to be injected may be spherical and may be required to have a long residence time in the bloodstream, i.e., avoid sequestration by the liver and other non-targeted organs. Bioprobe 390 may be successful in avoiding sequestration if its total diameter is less than about 30 nm. If bioprobe 390 contains a magnetite (Fe₃O₄) particle 342, then a diameter of susceptor 342 may be between about 8 nm and about 20 nm. In this case, bioprobes 390 may be sufficiently small to evade the liver, and yet the magnetic particle 342 still retains a sufficient magnetic moment for heating in an applied AMF. Magnetite particles larger than about 8 nm generally tend to be ferrimagnetic and thus appropriate for disease treatment. If other elements, such as cobalt, are added to the magnetite, this size range can be smaller. This results directly from the fact that cobalt generally possesses a larger magnetic moment than magnetite, which contributes to the overall magnetic moment of cobalt-containing susceptor 342. In general, the size of bioprobe 390 may be about 0.1 nm to about 250 nm, depending upon the disease indication and bioprobe composition.

Examples of susceptors for use herein include iron oxide particles and FeCo/SiO₂ particles. Some susceptors have a specific absorption rate (SAR) of about 310 Watts per gram of particle at 1,300 Oerstedt flux-density and 150 kHz frequency, such as series EMG700 and EMG1111 iron oxide particles of about 110 nm diameter available from Ferrotec Corp. (Nashua, N.H.). Other particles have a SAR of about 400 Watts per gram of particle under the same magnetic field conditions, such as the FeCo/SiO₂ particles available from Inframat Corp. (Willington, Conn.).

While determining the size of susceptor 342, its material composition may be determined based on the particular target. Because the self-limiting temperature of a magnetic material, or the Curie temperature, is directly related to the material composition, as is the total heat delivered, magnetic particle compositions may be tuned to different tissue or target types. This may be required because each target type, given its composition and location within the body, possesses unique heating and cooling capacities. For example, a tumor located within a region that is poorly supplied by blood and located within a relatively insulating region may require a lower Curie temperature material than a tumor that is located near a major blood vessel. Targets that are in the bloodstream will require different Curie temperature materials as well. Thus, in addition to magnetite, particle compositions may contain elements such as cobalt, iron, rare earth metals, etc.

The presence of coating 344 and the composition of the coating material may form an integral part of the energy loss, and thus the heat produced, by bioprobes 390. In addition, coating 344 may serve additional purposes. The coating 344 does not have to cover the whole bioprobe core 342, but may only partially cover the core 342. Coating 344 may provide a biocompatible layer separating the magnetic material from the immunologic defenses in a patient, thereby controlling the residence time of the particles in the blood or tissue fluids.

This control of residence time allows one to choose targeting ligands 340 that are best suited for a particular tissue type. In addition, coating 344 may serve to protect the patient from potentially toxic elements in susceptor 342. A second function of the coating materials may be the prevention of particle aggregation, as bioprobes 390 may be suspended in a fluid. It may be also be advantageous to coat bioprobe 390 with a biocompatible coating that is biodegradable or resorbable. In such an application, both the coating 344 and the susceptor 342 may be digested and absorbed by the body.

Suitable materials for the coating 344 include synthetic and biological polymers, copolymers and polymer blends, and inorganic materials. Polymer materials may include acrylates, siloxanes, styrenes, acetates, alkylene glycols, alkylenes, alkylene oxides, parylenes, lactic acid, glycolic acid, and combinations thereof. Further suitable coating materials include a hydrogel polymer, a histidine-containing polymer, and a combination of a hydrogel polymer and a histidine-containing polymer.

Coating materials may include biological materials such as polysaccharides, polyaminoacids, proteins, lipids, glycerols, fatty acids, and combinations thereof. Other biological materials for use as a coating material may include heparin, heparin sulfate, chondroitin sulfate, chitin, chitosan, cellulose, dextran, alginate, starch, carbohydrate, and glycosaminoglycan. Proteins may include an extracellular matrix protein, proteoglycan, glycoprotein, albumin, peptide, and gelatin. These materials may also be used in combination with any suitable synthetic polymer material.

Inorganic coating materials may include any combination of a metal, a metal alloy, and a ceramic. Examples of ceramic materials include hydroxyapatite, silicon carbide, carboxylate, sulfonate, phosphate, ferrite, phosphonate, and oxides of Group IV elements of the Periodic Table of Elements. These materials may form a composite coating that also contains biological or synthetic polymers. Where susceptor 342 is formed from a magnetic material that is biocompatible, the surface of the particle itself operates as the biocompatible coating.

The coating 344 material may also serve to facilitate transport of bioprobe 390 into a cell, a process known as transfection. Such coating materials, known as transfection agents, may include vectors, prions, polyaminoacids, cationic liposomes, amphiphiles, non-liposomal lipids, or any combination thereof. A suitable vector may be a plasmid, a virus, a phage, a viron, or a viral coat. The bioprobe coating may be a composite of a combination of transfection agents with organic and inorganic materials, such that the particular combination may be tailored for a particular type of a diseased cell and a specific location within a patient's body.

To ensure that bioprobe 390 selectively attaches to, or otherwise associates with, the target, an appropriate ligand 340 may be combined with bioprobe 390. The association of a ligand or ligands with bioprobes 390 allows for targeting of cancer or disease markers on cells. It also allows for targeting biological matter in the patient The term ligand relates to compounds which may target molecules including, for example, proteins, peptides, antibodies, antibody fragments, saccharides, carbohydrates, glycans, cytokines, chemokines, nucleotides, lectins, lipids, receptors, steroids, neurotransmitters, Cluster Designation/Differentiation (CD) markers, imprinted polymers, and the like. Examples of protein ligands include cell surface proteins, membrane proteins, proteoglycans, glycoproteins, peptides, and the like. Example nucleotide ligands include complete nucleotides, complimentary nucleotides, and nucleotide fragments. Example lipid ligands include phospholipids, glycolipids, and the like. Ligand 340 may be covalently bonded to or physically interacted with susceptor 342 or coating 344. Ligand 340 may be bound covalently or by physical interaction to an uncoated portion of susceptor 342. Ligand 340 may be bound covalently or by physical interaction directly to an uncoated portion of susceptor 342 and partially covered by coating 344. Ligand 340 may be bound covalently or by physical interaction to a coated portion of bioprobe 390. Ligand 340 may be intercalated to the coated portion of bioprobe 390.

Covalent bonding may be achieved with a linker molecule. The term “linker molecule”, as used herein, refers to an agent that targets particular functional groups on ligand 340 and on susceptor 342 or coating 344, and thus forms a covalent link between ligand 340 and susceptor 342 or coating 344. Examples of functional groups used in linking reactions include amines, sulfhydryls, carbohydrates, carboxyls, hydroxyls, and the like. The linking agent may be a homobifunctional or heterobifunctional crosslinking reagent, for example, carbodiimides, sulfo-NHS esters linkers, and the like. The linking agent may also be an aldehyde crosslinking reagent, such as glutaraldehyde. The linking agent may be chosen to link ligand 340 to susceptor 342 or coating 344 in a preferable orientation, specifically with the active region of the ligand 340 available for targeting. Physical interaction does not require that the linking molecule and ligand 340 be bound directly to susceptor 342 or to coating 344 by non-covalent means such as, for example, absorption, adsorption, or intercalation.

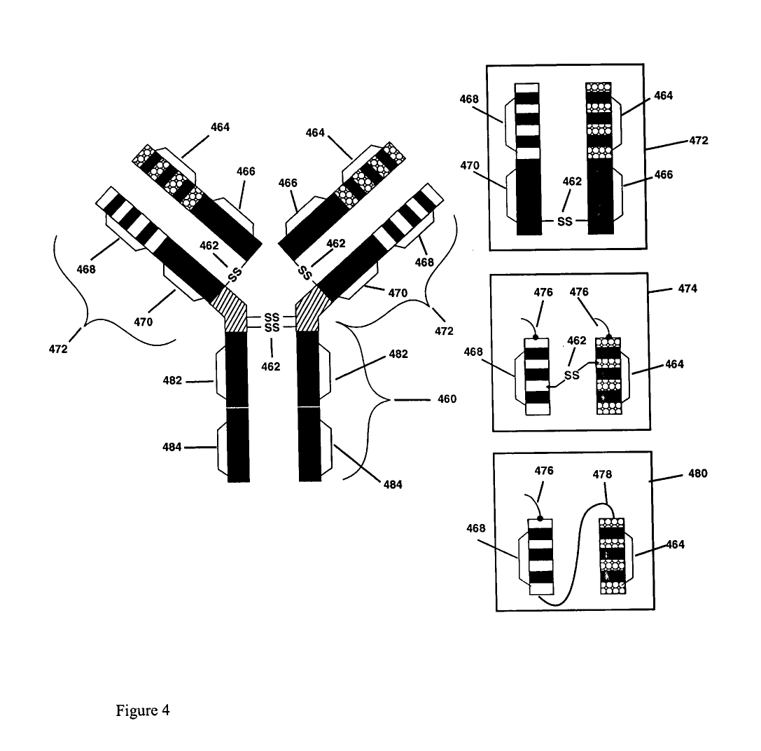

FIG. 4 schematically illustrates an example of a ligand that may be used with an embodiment of the present invention. The ligand may be an antibody having a fragment crystallization (Fc) region 460 and fragment antigen binding (Fab) regions 472. Fab regions 472 may be the antigen binding regions of the antibody that include a variable light region 464 and a constant light region 466, along with a variable heavy region 468 and a constant heavy region 470. Biological activity of antibodies may be determined to a large extent by the Fc region 460 of the antibody molecule. Fc region 460 may include complement activation constant heavy chains 482 and macrophage binding constant heavy chains 484. Fc region 460 and Fab regions 472 may be connected by several disulfide linkages 462. Ligands that do not include the Fc region 460 may be preferable in order to avoid immunogenic response. Examples of these ligands may include antibody fragments, fragment antigen binding fragments (Fabs) 472, disulfide-stabilized variable region fragments (dsFVs) 474, single chain variable region fragments (scFVs) 480, recombinant single chain antibody fragments, and peptides.

An antigen binding fragment (Fab) 472 may include a single Fab region 472 of an antibody. Single Fab region 472 may include a variable light 464 and a constant light region 466 bound to a variable heavy 468 and a constant heavy region 470 by a disulfide bond 462. A disulfide-stabilized variable region fragment (dsFV) 474 may include a variable heavy region 468 and a variable light region 464 of antibody joined by a disulfide bond. A leader sequence 476, which may be a peptide, may be linked to a variable light region 464 and variable heavy regions 468. Single chain variable region fragment (scFV) 480 may include a variable heavy region 468 and variable light region 464 of antibody joined by a linker peptide 478. A leader sequence 476 may be linked to the variable heavy region 468.

Examples of ligand embodiments of the present invention may include, for example, polyclonal antibodies, monoclonal antibodies, chimeric antibodies, humanized antibodies, human antibodies, recombinant antibodies, bispecific antibodies, antibody fragments, scFVs 480, Fabs 472, dsFVs 474, recombinant single chain antibody fragments, peptides, and the like. Bispecific antibodies are non-natural antibodies that bind two different epitopes that are typically chosen on two different antigens. A bispecific antibody is typically comprised of two different fragment antigen binding regions (Fabs) 472. A bispecific antibody may be formed by cleaving an antibody into two halves by cleaving disulfide linkages 462 in Fc region 482 only. Two antibody halves with different Fab regions 472 are then combined to form a bispecific antibody with the typical “Y” structure. One or more ligands can be present in the bioprobe formulation. Antibodies of varying origin may be used according to this embodiment, provided they bind the target, although human, chimeric, and humanized antibodies may aid in avoiding the patient's immunogenic response.

The choice of a marker (antigen) is useful in therapy utilizing bioprobes. For breast cancer and its metastases, a specific marker or markers may be chosen from cell surface markers such as, for example, members of the MUC-type mucin family, an epithelial growth factor (EGFR) receptor, a carcinoembryonic antigen (CEA), a human carcinoma antigen, a vascular endothelial growth factor (VEGF) antigen, a melanoma antigen (MAGE) gene, family antigen, a T/Tn antigen, a hormone receptor, growth factor receptors, a cluster designation/differentiation (CD) antigen, a tumor suppressor gene, a cell cycle regulator, an oncogene, an oncogene receptor, a proliferation marker, an adhesion molecule, a proteinase involved in degradation of extracellular matrix, a malignant transformation related factor, an apoptosis related factor, a human carcinoma antigen, glycoprotein antigens, DF3, 4F2, MGFM antigens, breast tumor antigen CA 15-3, calponin, cathepsin, CD 31 antigen, proliferating cell nuclear antigen 10 (PC 10), and pS2.

For other forms of cancer and their metastases, a specific marker or markers may be selected from cell surface markers such as, for example, vascular endothelial growth factor receptor (VEGFR) family, a member of carcinoembryonic antigen (CEA) family, a type of anti-idiotypic mAB, a type of ganglioside mimic, a member of cluster designation/differentiation antigens, a member of epidermal growth factor receptor (EGFR) family, a type of a cellular adhesion molecule, a member of MUC-type mucin family, a type of cancer antigen (CA), a type of a matrix metalloproteinase, a type of glycoprotein antigen, a type of melanoma associated antigen (MAA), a proteolytic enzyme, a calmodulin, a member of tumor necrosis factor (TNF) receptor family, a type of angiogenesis marker, a melanoma antigen recognized by T cells (MART) antigen, a member of melanoma antigen encoding gene (MAGE) family, a prostate membrane specific antigen (PMSA), a small cell lung carcinoma antigen (SCLCA), a T/Tn antigen, a hormone receptor, a tumor suppressor gene antigen, a cell cycle regulator antigen, an oncogene antigen, an oncogene receptor antigen, a proliferation marker, a proteinase involved in degradation of extracellular matrix, a malignant transformation related factor, an apoptosis-related factor, and a type of human carcinoma antigen.

In one embodiment of the present invention, the bioprobe attaches to, or associates with, cancer cells and is exposed to the AMF. Heat that is generated will destroy or otherwise deactivate immediately or over time (e.g., apoptosis) the cancer cells, which will be absorbed or otherwise removed from the body. In addition, cells that die by apoptosis will express and release heat shock proteins, such as HSP70, the presence of which can stimulate an immune reaction against any remaining cancer cells. Such a stimulated immune response may serve to protect the individual from future developments of cancer.

In another embodiment, ligand 340 (FIG. 3) may be targeted to a predetermined target associated with a disease of the patient's immune system. The particular target and ligand 340 may be specific to, but not limited to, the type of the immune disease. Ligand 340 may have an affinity for a cell marker or markers of interest. The marker or markers may be selected such that they represent a viable target on T cells or B cells of the patient's immune system. The ligand 340 may have an affinity for a target associated with a disease of the patient's immune system such as, for example, a protein, a cytokine, a chemokine, an infectious organism, and the like.

In another embodiment, ligand 340 may be targeted to a predetermined target associated with a pathogen-borne condition. The particular target and ligand 340 may be specific to, but not limited to, the type of the pathogen-borne condition. A pathogen is defined as any disease-producing agent such as, for example, a bacterium, a virus, a microorganism, a fungus, and a parasite. Ligand 340 may have an affinity for the pathogen or pathogen associated matter. Ligand 340 may have an affinity for a cell marker or markers associated with a pathogen-borne condition. The marker or markers may be selected such that they represent a viable target on infected cells.

For a pathogen-borne condition, ligand 340 may be selected to target the pathogen itself. For a bacterial condition, a predetermined target may be the bacteria itself, for example, Escherichia coli or Bacillus anthracis. For a viral condition, a predetermined target may be the virus itself, for example, Cytomegalovirus (CMV), Epstein-Barr virus (EBV), a hepatitis virus, such as Hepatitis B virus, human immunodeficiency virus, such as HIV, HIV-1, or HIV-2, or a herpes virus, such as Herpes virus 6. For a parasitic condition, a predetermined target may be the parasite itself, for example, Trypanasoma cruzi, Kinetoplastid, Schistosoma mansoni, Schistosoma japonicum or Schistosoma brucei. For a fungal condition, a predetermined target may be the fungus itself, for example, Aspergillus, Cryptococcus neoformans or Rhizomucor.

In another embodiment, the ligand 340 may be targeted to a predetermined target associated with an undesirable target. The particular target and ligand 340 may be specific to, but not limited to, the type of the undesirable target. An undesirable target is a target that may be associated with a disease or an undesirable condition, but also present in the normal condition. For example, the target may be present at elevated concentrations or otherwise be altered in the disease or undesirable state. Ligand 340 may have an affinity for the undesirable target or for biological molecular pathways related to the undesirable target. Ligand 340 may have an affinity for a cell marker or markers associated with the undesirable target.

For an undesirable target, the choice of a predetermined target may be important to therapy utilizing bioprobes. Ligand 340 may be selected to target biological matter associated with a disease or undesirable condition. For arteriosclerosis, a predetermined target may be, for example, apolipoprotein B on low density lipoprotein (LDL). For obesity, a predetermined marker or markers may be chosen from cell surface markers such as, for example, one of gastric inhibitory polypeptide receptor and CD36 antigen. Another undesirable predetermined target may be clotted blood.

In another embodiment, ligand 340 may be targeted to a predetermined target associated with a reaction to an organ transplanted into the patient. The particular target and ligand 340 may be specific to, but not limited to, the type of organ transplant. Ligand 340 may have an affinity for a biological molecule associated with a reaction to an organ transplant. Ligand 340 may have an affinity for a cell marker or markers associated with a reaction to an organ transplant. The marker or markers may be selected such that they represent a viable target on T cells or B cells of the patient's immune system.

In another embodiment, ligand 340 may be targeted to a predetermined target associated with a toxin in the patient. A toxin is defined as any poison produced by an organism including, but not limited to, bacterial toxins, plant toxins, insect toxin, animal toxins, and man-made toxins. The particular target and ligand 340 may be specific to, but not limited to, the type of toxin. Ligand 340 may have an affinity for the toxin or a biological molecule associated with a reaction to the toxin. Ligand 340 may have an affinity for a cell marker or markers associated with a reaction to the toxin.

In another embodiment, ligand 340 may be targeted to a predetermined target associated with a hormone-related disease. The particular target and ligand 340 may be specific to, but not limited to, a particular hormone disease. Ligand 340 may have an affinity for a hormone or a biological molecule associated with the hormone pathway. Ligand 340 may have an affinity for a cell marker or markers associated with the hormone disease.

In another embodiment, the ligand 340 may be targeted to a predetermined target associated with non-cancerous diseased tissue. The particular target and ligand 340 may be specific to, but not limited to, a particular non-cancerous diseased tissue, such as non-cancerous diseased deposits and precursor deposits. Ligand 340 may have an affinity for a biological molecule associated with the non-cancerous diseased tissue. Ligand 340 may have an affinity for a cell marker or markers associated with the non-cancerous diseased tissue.

In another embodiment, the ligand 340 may be targeted to a proteinaceous pathogen. The particular target and ligand 340 may be specific to, but not limited to, a particular proteinaceous pathogen. Ligand 340 may have an affinity for a proteinaceous pathogen or a biological molecule associated with the proteinaceous pathogen. Ligand 340 may have an affinity for a cell marker or markers associated with the proteinaceous pathogen. For prion diseases, also known as transmissible spongiform encephalopathies, a predetermined target may be, for example, Prion protein 3F4.

Some exemplary embodiments of the bioprobe system, along with associated indications for which they may be utilized, are listed in Table I.

TABLE I
BIOPROBE SYSTEMS AND INDICATIONS

| BIOPROBE SYSTEM | | | |
|---|---|---|---|
| TARGET | MARKER | LIGAND | INDICATION |
| Endothelial cells of | Integrin αvβ3 | Ber EP4 antibody | Metastatic breast cancer, metastatic |
| growing blood | | LM609 antibody | colon carcinoma |
| vessels of | | Integrin antagonist | |
| metastatic cancer | | | |
| cells | | | |
| Cancer cells | Unglycosylated DF3 | Anti-DF3 antibody | Breast cancer |
| | antigen | | |
| Cancer cells | Kallikreins | Anti-kallikrein | Ovarian and prostate cancer |
| | | antibody | |
| Cancer cells | ErbB2 (HER-2/neu) | Anti-ErbB2 antibody, | Breast and ovarian cancers |
| | | and scFv (F5), IDM-1 | |
| | | (aka MDX-210) | |
| | | variants | |
| Cancer cells | Prostate specific | MDX-070 and 7E11- | Prostate cancer |
| | membrane antigen | C5.3 antibodies | |
| | (PSMA) | | |
| MCF-7 breast | 43 Kd membrane | 323/A3 antibody | Breast cancer |
| cancer cells | associated | | |
| | glycoprotein | | |
| Receptor tyrosine | Vascular endothelial | Anti-FLT1 antibody | Tumour angiogenesis |
| kinases-- | growth factor | Anti-FLK1 antibody, | Tumour angiogenesis |
| FLT1 | (VEGF) and VEGFB | 2C3 antibody | |
| FLK1 | and placental growth | | |
| | factor receptors | | |
| | (PGFR) | | |
| Metastatic cancer | CAR (coxsackie | Anti-CAR antibody | Metastatic prostate cancer |
| cells | adenovirus cell- | | |
| | surface receptor) | | |
| Vascular smooth | Urokinase type | Urokinase type | Cancer |
| muscle cells of | plasminogen | plasminogen activator | |
| cancer cells | activator receptor | (uPA) | |
| | (uPAR) | | |
| Blood vessels of | Plasminogen | Anti-PAI-1 antibody | Breast cancer |
| cancer cells | activator inhibitor | | |
| | 1(PAI-1) | | |
| Epithelial ovarian | Matrix | Anti-MMP-9 antibody | Ovarian carcinomas with lymph |
| tumour cells | metaloproteinase 9 | | node metastasis. |
| | (MMP-9) | | |
| Cancer cells | Cyclin A | Anti-cyclin A antibody | Squamous cell carcinoma of the |
| | | | tongue |
| Cancer cells | Cyclin D | Anti-cyclin D(1, 2, 3) | Malignant breast cancer, head and |
| | | antibody | neck squamous cell carcinomas, |
| | | | mantle cell carcinomas, laryngeal |
| | | | squamous cell carcinomas |
| Kidney cortex tissue | Cyclin E | Anti-cyclin E antibody | Human renal cell carcinoma |
| Tumorigenic human | Cyclin E | Anti-cyclin E antibody | Breast cancer |
| breast epithelial | | | |
| cells | | | |
| Malignant epithelial | Cyclin E | Anti-cyclin E antibody | Transitional cell carcinoma of the |
| bladder tissue | | | urinary bladder |
| Cancer cells | Cdc 2 | Anti-cdc 2 antibody | Breast cancer |
| Malignant epithelial | P27 | Anti-phospho p27 | Transitional cell carcinoma of the |
| bladder tissue | | antibody | urinary bladder |
| Cancer cells | P73 | Anti-p73 antibody | Lung carcinogenesis, bladder |
| | | | carcinogenesis, neuroblastoma, |
| | | | breast cancer |
| Cancer cells | Ras | Anti-ras antibody | Breast cancer |
| Cancer cells | c-myc | Anti C-myc antibody | Breast cancer |
| Cancer cells | c-fms | Anti-c-fms antibody | Breast cancer |
| Cancer cells | Hepatocyte growth | Anti-HGFR antibody | Colorectal cancer |
| | factor receptor | | |
| | (HGFR) | | |
| Cancer cells | c-met | Anti-c-met antibody | Gastric and colon cancers, |
| | | | hepatomas, ovarian cancer, skin |
| | | | cancer |
| Large granular | Apoptosis related | Anti-CD95 (Fas) | Leukaemia, prostate cancer |
| lymphocyte (LGL) | factors: | antibody | |
| leukaemia cells | Fas | | |
| | FasL | | |
| Cancer cells | Non-receptor protein | Anti c-src-polyclonal | Metastatic colorectal cancer, and |
| | tyrosine kinase V- | antibody | late stage breast cancer |
| | Src and C-Src | | |
| Cancer cell | CAR (coxsackie | Onyx-015 adenovirus | Lung, ovarian, other cancers |
| | adenovirus cell- | | |
| | surface receptor) | | |
| Cancer cell | Epidermal growth | Molecule 225 antibody | Cancer |
| | factor receptor | | |
| | (EGFR) | | |
| Cancer cells | D6 antigen | Anti-D6 antibody | Vascular tumours including |
| | | | Kaposi's sarcoma |
| Cancer cells | 2C4 antigen | Anti-2C4 antibody | Breast, prostate, other cancers |
| Cancer cells | Cytokeratin | S5A10-2 antibody | Non-small cell lung cancer |
| | epithelial marker | | |
| | and/or telomerase | | |
| | reverse transcriptase | | |
| Cancer cells | Carcinoembryonic | MFE-23 scFv of anti- | Colorectal cancer |
| | antigen (CEA) | CEA antibody | |
| Cancer cells | Proliferating cell | Anti-PCNA antibody | Breast cancer |
| | nuclear antigen | | |
| | (PCNA) | | |
| Cancer cells | Neu 3, a membrane | Anti-neu 3 sialidase | Colon cancer |
| | associated sialidase | antibody | |
| Cancer cells | P13KC2 beta (cancer | Anti-P13KC2beta | Lung cancer |
| | cell signal mediator) | antibody | |
| Cancer cells | Guanylyl cyclase-C | Anti-GC-C antibody | Esophageal or gastric cancer |
| | (GC-C) receptor | | |
| Cancer cells | Transforming | Anti-TGFB antibody | Breast cancer |
| | growth factor beta | | |
| | (TGFB) receptor | | |
| Cancer cells | Platelet derived | Anti-PDGF-A | Lung cancer |
| | growth factor | antibody | Bone cancer |
| | receptor (PDGFR) | Anti-PDGF-B antibody | |
| | PDGFR-A (alpha) | | |
| | PDGFR-B (beta) | | |
| Cancer cells and | Vascular endothelial | Tie1 | Cancer |
| blood vessels | growth factors | Tie2 | Cancer |
| | VEGFR | | |
| | Angiopoietin | | |
| Cancer cells | Mucin family of | Anti-MUC-1 antibody, | Colorectal and ovarian carcinomas |
| | receptors | 12E antibody | |
| | | 3D antibody | |
| | | A5 antibody | |
| Cancer cells | TAG-72 | B72.3 antibody | Breast and lung cancers |
| Cancer cells | Human milk fat | NCL-HMFG1 and | Breast, lung, colon, and prostate |
| | globule receptor | NCL-HMFG2 | cancers |
| | | antibodies | |
| Methionine synthase | Cobalamin receptor | B12 (riboflavin, and | Breast, lung, colon, sarcomatous |
| and L- | | variants) cobalamin | thyroid or central nervous system |
| methylmalonyl-CoA | | and variants such as | malignancies cancer |
| mutase | | adenosylcobalamin | |
| | | transcobalamin | |
| Cancer cells | Glioma chloride | Scorpion toxin— | Gliomas |
| | channel | chlorotoxin and | |
| | | chlorotoxin-like | |
| | | molecules | |
| Cancer cells | 40 kD glycoprotein | NR-LU-10 antibody | Small cell lung cancer |
| | antigen | | |
| CNS cells and tissue | Brain-specific | Anti-BEHAB antibody | Gliomas |
| | chondroitin sulphate | | |
| | proteoglycan | | |
| | Brain enriched | | |
| | hyaluronan binding | | |
| | protein (BEHAB- | | |
| | aka brevican | | |
| Cancer cells | Catenins | Anti-alpha catenin | Colorectal carcinoma, non-small |
| | Alpha catenin | antibody | cell lung cancer |
| | Beta catenin | Anti-beta catenin | Breast cancer |
| | Gamma catenin | antibody | Thyroid cancer |
| | | Anti-gamma catenin | |
| | | antibody | |
| Cancer cells | Interleukin (IL) | IL13-PE38 antibody | Kidney, brain, breast, and head and |
| | receptors | | neck cancers, and Kaposi's sarcoma |
| | IL13 receptor | | |
| Cancer cells | Mesothelin receptor | Anti-mesothelin | Mesotheliomas |
| | | antibody, and | Ovarian cancer and mesotheliomas |
| | | SS1(dsFv) variant | |
| Cancer cells | CD44 surface | Anti-CD44 antibody | Prostate cancer |
| | adhesion molecule | | |
| Cancer cells | EGFRvIII | Ua30:2 antibody | Brain, colorectal, pancreatic, billary, |
| | | L8A4 antibody | liver cancers and soft tissue |
| | | DH8.3 antibody | sarcomas. |
| | | 81C6 antibody | |
| Receptor tyrosine | Vascular endothelial | Anti-FLT1 antibody | Atherosclerotic plaques |
| kinases FLT1 | growth factor | | |
| | (VEGF) and VEGFB | | |
| Smooth muscle cells | Basic fibroblast | Anit-bFGF antibody | Restenosis |
| in the lumen of | growth factor | | |
| blood vessels | receptor (bFGFR) | | |
| Vulnerable plaque | Oxidized low density | Oxidation-specific | Atherosclerosis and vascular disease |
| | lipoprotein (OxLDL) | antibodies (Ox-AB) | |
| | | MDA-2 antibody | |
| Vulnerable plaque | Malondialdehyde- | 1K17 antibody | Atherosclerosis and vascular disease |
| | modified LDL | | |
| | (MDA-LDL) | | |
| M. Tuberculosis | APA-antigen | Anti-APA antibody | Tuberculosis |
| bacilli | | | |
| Retrovirus infected | TGFA (alpha) | Anti-TGFA antibody | HIV |
| cells | | | |
| Leukocytes | Alpha4 subunit of | Antegren | Multiple sclerosis |
| | alpha4beta1-integrin | | |
| | (VLA-4) and | | |
| | alpha4beta7-integrin | | |
| Receptor tyrosine | Vascular endothelial | Anti-FLT1 antibody | Autoimmune joint destruction |
| kinases FLT1 | growth factor | | (arthritis, lupus, etc) |
| | (VEGF) and VEGFB | | |
| Plasmodium | Apical membrane | Anti-AMA-1 antibody | Malaria |
| falciparum | antigen-1 (AMA-1) | | |
| Cells of the immune | CD30 | AC10, HeFil, and | Immunological disorders other than |
| system | | derivatives of AC10 | cancer |
| | | and HeFil | |
| Hepatitis C virus | Hepatitis C virus | 19D9D6 Monoclonal | Hepatitis C infection |
| | core protein | Antibody | |
| Tumor vascular cells | Vascular endothelial | MV833 and HuMV833 | Cancer |
| | growth factor | antibodies | |
| | (VEGF) | | |
| Tumor cells | Cytokeratin | Anti-cytokeratin | Epitheleoid sarcomas |
| | | AE1/3 and anti- | |
| | | CAM5.2 antibodies | |
| Tumor cells | Thomsen | M170, chimeric M170, | Breast, Prostate, Ovarian, and Lung |
| | Friedenreich (TF) | MaB 170H.82R1808 | cancers |
| | antigen | | |
| Tumor cells | CEA | HumaSpect ™, | Colon and Ovarian cancers |
| | | Votumumab, | |
| | | Mab 88BV59 | |
| Tumor cells | EFG-r | ABX-EGF | Colon, NSCLC, Prostate, and |
| | | | Renal cancers |
| Tumor cells | EGF-r | HuMax-EGFr | Head, Neck, Breast, Colon, |
| | | | Prostate, Lung, and Ovarian |
| | | | cancers |
| Tumor cells | EGF-r | TheraCIM ™, h-R3 | Head and Neck cancers |
| Tumor cells | CEA | KSB309 ™ | Oral cavity, and Pharngial cancers |
| Tumor cells | CEA | 4B5-H | Melanoma |
| Tumor cells | GD2 ganglioside | ABX-MA1 | Melanoma, Neuroblastoma, NSCLC |
| Tumor cells | CTLA4; CD152 | MDX-010 | Melanoma |
| Tumor cells | GD2 ganglioside | TriGem, Mab-1A7 | Melanoma |
| Tumor cells | CA125; MUC-16 | ACA-125 | Ovarian cancer |
| Tumor cells | Polymorphic | R1549, Pemtumomab, | Ovarian, Stomach, Breast, Lung, |
| | epithelial mucin | MuHMFg1, HuHMFg1 | and Prostate cancers |
| Tumor cells | CA125 | OvaRex ™, Mab- | Ovarian cancer |
| | | B42.13, Ov | |
| Tumor cells | | VB2-011, H-11 ScFv, | Breast, Ovarian, and Colorectal |
| | | Novo Mab-G2ScFv | cancers |
| Tumor cells | CEA | CEA-Cide, | Breast, Colon, and Lung cancers |
| | | Labetuzumab | |
| Tumor cells | VEGF | Avastin ™, | Breast, Colorectal, NSCLC, and |
| | | Bevacizumab, | Renal cancers |
| | | rhuMAb-VEGF | |
| Tumor cells | LewisY Ag | SGN-15, cBR96 | Breast, NSCLC, and Ovarian |
| | | | cancers |
| Tumor cells | HER2 | OmniTag ™, | Breast, Ovarian, Lung, and Prostate |
| | | Pertuzumab, | cancers |
| | | rhuMAb 2C4 | |
| Tumor cells | MUC1 | BrevaRex ™, Mab | Breast, Ovarian, and Multiple |
| | | AR20.5 | Myeloma cancer |
| Tumor cells | MUC1 | Therex ™, R1550, | Breast, Ovarian, Pancreatic, and |
| | | HuHMFG1 | Gastric cancers |
| Tumor cells | Ep-CAM | ING-1 | Breast, Lung, Prostate, and |
| | | | Pancreatic cancers |
| Tumor cells | αvβ3 integrin | Vitaxin ™, huLM609 | Solid tumors |
| Tumor cells | αvβ3 integrin | Mab-MEDI-522, | Advanced solid tumors |
| | | huLM609 | |

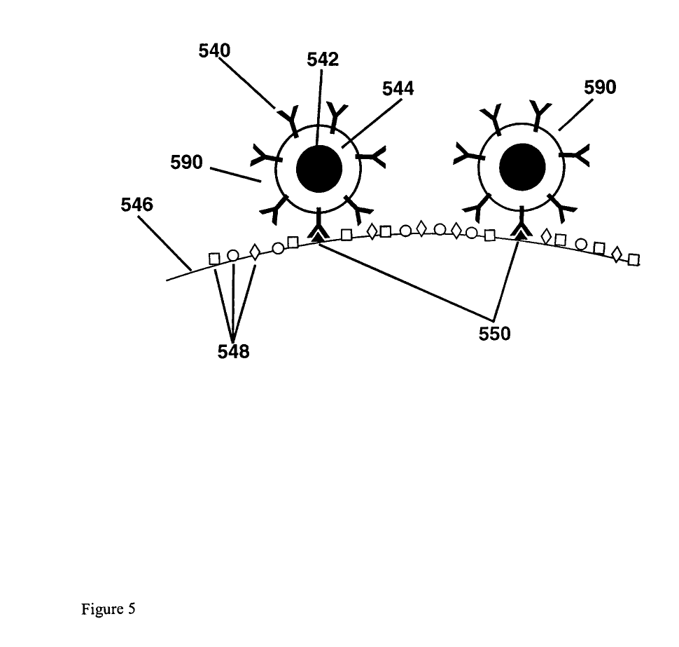

FIG. 5 illustrates an embodiment of the present invention wherein a bioprobe 590, comprising a susceptor 542, which comprises a coating 544, is attached to or associated with a target (such as a cell) 546 by one or more targeting ligands 540. Cell 546 may express several types of markers 548 and 550. The specificity of bioprobe 590 is represented by its attachment to targeted marker 550 over the many other markers or molecules 548 on cell 546. One or more bioprobes 590 may attach to or associate with cell 546 using ligand 540. Ligand 540 may be adapted and bioprobe 590 may be designed such that bioprobe 590 remains externally on cell 546 or may be internalized into cell 546. Once bound to cell 546, the susceptor 542 is energized in response to the energy absorbed. For example, the susceptor 542 may heat up in response to the energy absorbed. The heat may pass through coating 544 or through interstitial regions to the cell 546, for example by convection, conduction, radiation, or a combination of these heat transfer mechanisms. The heated cell 546 becomes damaged, preferably in a manner that causes irreparable damage. When bioprobe 590 becomes internalized within cell 546, bioprobe 590 may heat cell 546 internally via convection, conduction, radiation, or a combination of these heat transfer mechanisms. When a sufficient amount of energy is transferred by bioprobe 590 to cell 546, cell 546 dies via necrosis, apoptosis, or another mechanism.

A method of administering bioprobes 590 to the desired area for treatment and the dosage may depend upon, but is not limited to, the type and location of the diseased material. The size range of bioprobes 590 allows for microfiltration for sterilization. An administration method may be, for example, wash, lavage, as a rinse with sponge, or other surgical cloth as a perisurgical administration technique. Other methods of administration include intravascular injection, intravenous injection, intraperitoneal injection, subcutaneous injection, and intramuscular injection. Bioprobes 590 may be formulated in an injectable format (suspension, emulsion) in a medium such as, for example, water, saline, Ringer's solution, dextrose, albumin solution, or oils. Bioprobes 590 may also be administered to the patient through topical application via a salve or lotion, transdermally through a patch, orally ingested as a pill or capsule or suspended in a liquid, or rectally inserted in suppository form. Bioprobes 590 may also be suspended in an aerosol or pre-aerosol formulation suitable for inhalation via the mouth or nose. Once administered to the patient, delivery of bioprobes 590 to the target site may be assisted by an applied static magnetic field due to the magnetic nature of the bioprobes. Assisted delivery may depend on the location of the target.

2.2. Single-Domain Particles

It is well known that a magnetic body is divided into uniformly magnetized regions (domains) separated by domain walls (Bloch walls) in order to minimize its magnetostatic energy. This type of magnetic structure is referred to as a multidomain structure. The energy to be minimized is the total energy, which is a sum of the magnetostatic, the exchange, and the anisotropy energies as well as the energy of the domain wall itself. Therefore, it is the final balance of energies that determines the domain structure and shape.

When the dimensions of the magnetic body, i.e. crystal, are reduced, the size of the domains is also reduced, and their structure, as well as the width and the structure of the domain walls, may change. Due to the cost of energy wall formation, the balance with the magnetostatic energy limits the subdivision in domains to a certain optimum domain size. Indeed, there is a corresponding lower limit of crystal size, below which only a single-domain structure can exist, since the energy increase due to the formation of domain walls is higher than the energy decrease obtained by dividing the single domain into smaller domains.

For typical magnetic materials, the dimensional limit is in the range of about 20-800 nm, depending on the spontaneous magnetization and on the anisotropy and exchange energies. The change from a multidomain to a single-domain structure is accompanied by a strong increase of the coercive field. Variations of the dimensional limit occur and are governed by material composition, material shape, and crystal properties such as anisotropy and exchange energies. Since material shape and crystal properties are in turn determined by the material processing and environmental conditions, i.e., sample history, it is impossible to categorically state single-domain dimensions for even a material composition. Thus, each sample must be individually characterized to determine the average domain structure.

Superparamagnetic Particles: The anisotropy energy in a single-domain particle is proportional, in a first approximation, to the volume V. For uniaxial anisotropy, the associated energy barrier, separating easy magnetization, directions of the crystal (i.e., the low-energy directions of the magnetization vector, or spin system) is E_{B}=KV. Thus, with decreasing particle size, the anisotropy energy decreases, and for a grain size lower than a characteristic value, it may become so low as to be comparable to or lower than the thermal energy kT. This implies that the energy barrier for magnetization reversal may be overcome, and then the total magnetic moment of the particle can thermally fluctuate, like a single spin in a paramagnetic material. Thus, the entire spin system may be rotated, the spins within the single-domain particles remaining magnetically coupled (ferromagnetically or antiferromagnetically). The magnetic behavior of an assembly of such ultrafine, independent magnetic particles is referred to as superparamagnetism. [For a discussion on superparamagnetism, also refer to J. L. Dormann, “Magnetic Relaxation in Fine-Particle Systems”, Advances in ChemicalPhysics, Vol. XCVIII, ISBN 0-471-16285-X, 1997, Wiley & Sons, Inc., page 283-494.]

Superparamagnetic behavior is exhibited by particles with dimensions in a defined range. If they are too small, almost all the atoms lie on the surface, leading to electronic and magnetic properties strongly modified with respect to the bulk properties, and the superparamagnetic model cannot be applied. This does not mean that no relaxation of the magnetic moment occurs, but the laws governing it are expected to be different. It is difficult to state precisely a lower dimensional limit for superparamagnetic behavior, as it depends on several parameters. In many cases, it is believed to be about 2 nm. As far as the upper limit is concerned, it is given in principle by the characteristic size for a single-domain particle, as long as the single-domain state and structure are effective (some uncertainties remain for some particular cases). Actually the characteristic grain size of a magnetic material for superparamagnetic relaxation depends on the anisotropy constants and magnetic saturation values. As an example, for uniaxial anisotropy and K=5×10⁵ erg/cm³, for spherical particles this corresponds to a characteristic diameter φ_{c}≦20 nm.

For fine magnetic particles the actual magnetic behavior depends not only upon the material and physical characteristics of the particles, but also on the value of the measuring time (τₘ) of the specific experimental technique with respect to the relation time (τ) associated with overcoming the energy barriers. The characteristic relaxation time, τ, varies exponentially with the E_{B}/kT ratio. If τₘ>>τ, the relaxation appears to be so fast that a time average of the magnetization orientation is observed in the experimental time window, and the assembly of particles behaves like a paramagnetic system, i.e., superparamagnetic behavior is observed and the sample appears to be in the superparamagnetic state. On the other hand, if τₘ<<τ, the relaxation appears so slow that quasi-static properties are observed (blocked state), as with magnetically ordered crystals, although strongly influenced by the particle surface structure.

The blocking temperature T_{B}, separating the two states, is defined as the temperature at which τₘ=τ. Therefore, T_{B} is not uniquely defined as well as φ_{c}, but is related to the time scale of the experimental technique. As an example, for Fe₃O₄ (K=4.4×10⁵ erg/cm³) at 290 K, the characteristic grain diameter for superparamagnetism, below which superparamagnetic relaxation and above which quasi-static properties are observed, is φ_{c}≅17 nm for DC susceptibility measurements, while it is φ_{c}≅9 nm for Mössbauer spectroscopy experiments, having a much shorter measuring time.

The blocking temperature T_{B} for a magnetic particle increases with increasing size and for a given size increases with decreasing measuring time, and then the observation of a superparamagnetic of blocked state depends on the experimental technique. The highest value of T_{B} is represented by the Curie (or Neel) temperature, at which the transition from the superparamagnetic to the paramagnetic state occurs. For magnetite, this is about 858 K. The techniques currently used to study the superparamagnetic relaxation are DC susceptibility, AC susceptibility, Mössbauer spectroscopy, ferromagnetic resonance, and neutron diffraction. Table II displays the time window associated with each measurement technique.

TABLE II
TECHNIQUES TYPICALLY USED TO
MEASURE MAGNETIC PROPERTIES OF ULTRAFINE
PARTICLES, AND THEIR TIME WINDOWS.

| | Time window | |
|---|---|---|
| Technique | (sec.) | Comments |
| DC susceptibility | 100 | Estimated, time |
| | | is not well defined. |
| AC susceptibility | 10²-10⁴ | Low frequency |
| | 10⁻¹-10⁻⁵ | Classical experiments |
| | 10⁻⁵-10⁻⁸ | Very high frequencies, |
| | | difficult to realize |
| Mossbauer spectroscopy | 10⁻⁷-10⁻⁹ | For ⁵⁷Fe |
| Ferromagnetic resonance | 10⁻⁹ | |
| Neutron diffraction | 10⁻⁸-10⁻¹² | Depends upon |
| | | type of experiment |

Complexity of Actual Fine-Particle Systems and Hysteretic Heating: The discussion above was restricted to idealized examples of magnetic ultrafine (nanometer-sized) particles. Unfortunately, the actual situation in materials consisting of fine particles is very complex, and it is often necessary to account for the simultaneous presence of different factors.

First, in actual systems, there is always a distribution of particle size. Moreover, different terms can contribute to the total anisotropy energy of a single-domain particle, for example magnetocrystallinity, magnetostatic, shape, stress, and surface. The surface, which is closely related to the detailed chemical nature of surface and grain boundary, may become the dominant contribution to the anisotropy energy for particles smaller than about 10 nm.

For the application considered in this disclosure, a suspension of magnetic nanometer-sized (may be single-domain) particles is surrounded by polymer to form a bioprobe. When this suspension is exposed to an externally applied alternating magnetic field of frequency f and magnitude H, the magnetic moments within each particle may respond by changing orientation to align with the imposed external field. When the field direction is reversed, the magnetic moments of the particles attempt to respond by reorienting with the changing field vector. The extent to which they are able to accomplish this, and the extent to which they must overcome their internal energies (described above) may result in the production of heat. The amount of heat released by the particles will depend upon the several factors governing both the orientation of the particle magnetic moment with respect to its easy axis in the crystal and the external field, shape, anisotropy constant, etc. Thus, application of a magnetic field for hysteretic heating may be considered as a magnetic sampling experiment since it possesses the relevant conditions of time scale and temperature necessary in magnetic characterization experiments (cf. Table I). Typically, the magnetic properties of suspensions of nanoparticles are characterized by techniques with time windows (and temperatures) that do not correspond to the conditions of the actual application for hysteretic heating. This discrepancy may lead to the mis-characterization of the particle as being superparamagnetic, as this is the behavior observed during magnetic characterization. But this characterization may not be consistent for the application because the conditions (temperature, time scale) employed during application may be very different, with the particles exhibiting blocked (or ferromagnetic) behavior. Thus, to characterize actual samples with the inherent variations of particle size, shape, magnetic crystalline energies, etc. based upon measurement conditions that do not correspond to conditions actually used for hysteretic heating may be erroneous.

2.3. Biomineralization and Magnetic Nanoparticles

Two fundamentally different modes of biomineralization can produce magnetic nanometer-sized particles. One is referred to as biologically induced mineralization (BIM), in which an organism modifies its local microenvironment creating conditions suitable for the chemical precipitation of extracellular mineral phases. The second mode is referred to as boundary organized biomineralization (BOB), in which inorganic particles are grown within or on some organic matrix produced by the organism.

Bacteria that produce mineral phases by BIM do not strictly control the crystallization process, resulting in particles with no unique morphology and a broad particle size distribution. Non-magnetotactic dissimilatory iron-reducing and sulfate-reducing bacteria produce magnetite, siderite, vivianite, and iron sulfides by BIM processes. For example, the iron-reducing bacterium Geobacter metallireducens (formerly GS-15) is a non-magnetotactic anaerobe that couples the oxidation of organic matter to the reduction of ferric iron, inducing the extracellular precipitation of fine-grained magnetite as a byproduct.

In contrast to BIM, bacteria that produce mineral phases by a BOB processes exert strict control over size, morphology, composition, position, and crystallographic orientation of the particles. One example of microorganisms using BOB process to produce iron biominerals is magnetotactic bacteria. These bacteria synthesize intracellular, membrane-bounded Fe₃O₄ (magnetite), Fe₃S₄ (possible Fe₇S₈) and FeS₂ particles called manetosomes. Various arrangements of magnetosomes within cells impart a permanent magnetic dipole moment to the cell, which effectively makes each cell a self-propelled biomagnetic compass.

The hallmarks of magnetosomes are their size specificity and distinctive crystal morphologies. Many magnetosomes fall within a size of about 35-120 nm when measured along their long axis. This size specificity of magnetosomes is significant because within this size range the particles are uniformly magnetized, permanent single magnetic domains.

For a given cell type, magnetosomes have a uniform size, shape, crystal morphology, and arrangement within the cell. Magnetosomes occur in at least three different crystal forms determined using transmission electron microscopy. The simplest form, found in Magnetospirillum magnetotacticum, is cubo-octahedral, which preserves the cubic crystal symmetry of magnetite. A second type, found in coccoid and vibrioid strains, is an elongated hexagonal prism with the axis of elongation parallel to the <111> crystal direction. A third type, observed in some uncultured cells, is an elongated cubo-octahedral form producing unique bullet-shaped, teardrop, and arrowhead particles.

The ability of these bacteria to produce precisely formed, single-domain magnetic particles may be valuable for the production of bioprobes. These cells can be grown in cell cultures to manufacture quantities of magnetic particles, which can then be harvested and further modified with biocompatible coating material and ligands to produce the bioprobes. In addition, molecular biology, gene sequencing and cloning techniques may be used to further modify the strains of bacteria to produce well-controlled single domain particles all with identical sizes and properties that are different from those observed in the natural state.

2.4. The Energy Source for the Targeted Thermotherapy System

The energy source for use in the present invention includes any device that is able to provide energy to the susceptor that can convert that energy, for example to heat or mechanical motion. The bioprobe then transmits the heat or mechanical motion to the targeted cell and cells or tissue surrounding the targeted cell. The different forms of energy, for example AMF, microwave, acoustic, or a combination thereof, may be created using a variety of heating mechanisms.

Induction heating is typically accomplished by using any one of many commercially available RF generators. These generators may comprise chopped DC with a resonant network, or a vacuum tube or solid-state oscillator with or without an amplification stage and with or without an impedance matching or transformation stage.

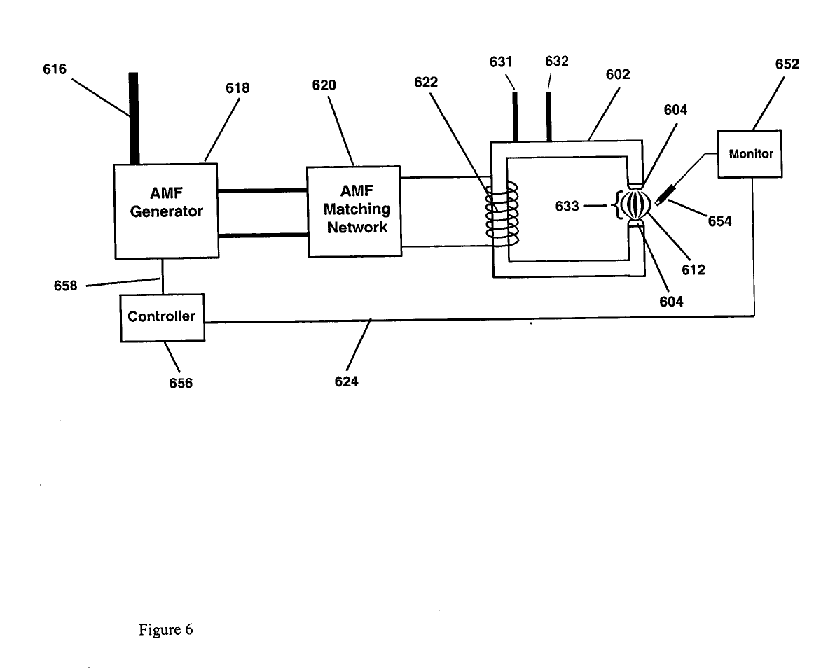

FIG. 6 illustrates a circuit for producing an AMF according to an embodiment of the present invention. An AMF generator 618 is supplied with alternating current (AC) power via a conduit 616. A circulating fluid supply is also provided in conduit 616. AMF generator 618 may become hot, and it may be cooled with the circulating fluid supply while in operation. The fluid may be water; however a fluid such as silicone oil or other inorganic or organic fluids with suitable thermal and electric properties may be preferable to increase generator efficiency. The energy produced by generator 618 is directed through an AMF matching network 620 where the impedance of the generator is matched to the impedance of a solenoid coil 622. The impedance of the AMF matching network 620 may be adjustable to minimize the energy reflected back to generator 618. In another embodiment, the generator frequency may be automatically adjusted to minimize the reflected energy. The modified energy may be directed to a magnetic circuit 602. An AMF is induced in magnetic circuit 602 as a result of the current passing through solenoid coil 622. Magnetic lines of flux 612 are produced in a gap 633 between the poles 604 in magnetic circuit 602. Liquid cooling send 631 and return 632 facilitate the cooling process.

A feedback loop 624 may be provided for monitoring the magnetic field profile in gap 633 between poles 604. A probe 654 may provide data to a monitor 652, which relays information to a controller 656 via an appropriate data bus 624. Information from controller 656 is relayed to generator 618 via an appropriate data bus 658. Monitoring the magnetic field profile may be useful in detecting the presence of magnetic particles, monitoring an inductance of tissue, and monitoring the temperature of tissue located in gap 633.

Measuring alternating magnetic fields directly is extremely difficult. Because the AMF is proportional to the current in solenoid coil 622, characteristics of the AMF may be defined in terms of the coil current, which can readily be measured with available test equipment. For example, the coil current may be viewed and measured with a calibrated Rogowski coil and any oscilloscope of suitable bandwidth. The fundamental waveform may be observed as the direct measure of the magnitude and direction of the coil current. Many different types of fundamental waveforms may be used for the AMF. The shape of the fundamental waveform may also be square, sawtooth, or trapezoidal.

Most practical generators produce an approximation of these waveforms with some amount of distortion. In most applications, this waveform may be nearly symmetrical around zero. However, there may be a static (DC) current, known as a DC offset, superimposed on the waveform. An AMF with a DC offset can be used to influence the movement of bioprobes within the body. With a suitable gradient and the “vibration-like” effect of the AC component, the bioprobes are typically drawn toward the area of highest field strength. The fundamental period may be defined as the time it takes to complete one cycle. The fundamental frequency may be defined as the reciprocal of the fundamental period. The fundamental frequency may be between 1 kHz and 1 GHz, preferably between 50 kHz and 15 MHz, and more preferably between 100 kHz and 500 kHz. The fundamental frequency may be intentionally modulated, and may often vary slightly as a result of imperfections in the RF generator design.

The amplitude of the waveform may also be modulated. The shape of the amplitude modulation envelope is typically sinusoidal, square, triangular, trapezoidal or sawtooth, however, it may be any variation or combination thereof, or may be some other shape.

The AMF produced by the generator may also be pulsed. Pulse width is traditionally defined as the time between the −3 dBc points of the output of a square law crystal detector. Because this measurement technique is cumbersome in this application, we use an alternate definition of pulse width. For the purpose of this invention, pulse width may be defined as the time interval between the 50% amplitude point of the pulse envelope leading edge and the 50% amplitude point of the pulse envelope trailing edge. The pulse width may also be modulated.

The pulse repetition frequency (PRF) is defined as the number of times per second that the amplitude modulation envelope is repeated. The PRF typically lies between 0.0017 Hz and 1000 MHz. The PRF may also be modulated. The duty cycle may be defined as the product of the pulse width and the PRF, and thus is dimensionless. In order to be defined as pulsed, the duty of the generator 618 must be less than 100%.

The AMF may be constrained to prevent heating healthy tissue to lethal temperatures, for example by setting the temperature of the tissue to be around 43° C., thus allowing for a margin of error of about 3° C. from the temperature of 46.5° C. that is lethal to healthy tissue. This may be accomplished in a variety of ways.
The peak amplitude of the AMF may be adjusted.
The PRF may be adjusted.
The pulse width may be adjusted.
The fundamental frequency may be adjusted.
The treatment duration may be adjusted.

These characteristics may be adjusted to maximize the heating rate of the bioprobes and, simultaneously, to minimize the heating rate of the healthy tissue located within the treatment volume. These conditions may vary depending upon tissue types to be treated, thus the operator may determine efficacious operation levels. In one embodiment, one or more of these characteristics may be adjusted during treatment based upon one or more continuously monitored physical characteristics of tissue in the treatment volume by probe 654, such as temperature or impedance. This information may then be supplied as input to generator 618, via monitor 652, data bus 624, controller 656, and data bus 658 to control output, constituting the feedback loop. In another embodiment, one or more physical characteristics of the bioprobes (such as magnetic properties) may be monitored during treatment with a suitable device. In this case, one or more magnetic property, such as the magnetic moment, is directly related to the temperature of the magnetic material. Thus, by monitoring some combination of magnetic properties of the bioprobe, the bioprobe temperature can be monitored indirectly. This information may also be supplied as input to generator 618, via monitor 652, data bus 624, controller 656, and data bus 658 to control output to become part of the feedback loop. The generator output may be adjusted so that the peak AMF strength is between about 10 and about 10,000 Oersteds (Oe). Preferably, the peak AMF strength is between about 20 and about 3000 Oe, and more preferably, between about 100 and about 2000 Oe.

In another embodiment of the present invention, the differential heating of the bioprobes, as compared to that of the healthy tissue, may be maximized. Bioprobes 210 (FIG. 2) heat in response to each cycle of the AMF. Assuming the fundamental frequency, the PRF, and the pulse width will remain constant, the heat output of bioprobe 210 continues to increase as peak amplitude of the AMF increases until the magnetic material of the bioprobe reaches saturation. Beyond this point, additional increases in AMF amplitude yield almost no additional heating. At AMF amplitudes below saturation however, it can be said that bioprobe heating is a function of AMF amplitude. Unlike bioprobes, healthy tissue heating is a result of eddy current flow and a function of the rate of change of the AMF.

In one embodiment of the present invention, a symmetrical triangular wave is the fundamental waveform of the AMF. By avoiding the high rates of change that occur as a sinusoid crosses the X-axis, and substituting the constant but lower rate of change associated with a triangular waveform, tissue heating may be reduced with little or no sacrifice in bioprobe heating. A triangular waveform may be achieved by using an appropriate generator, such as a linear amplifier-based generator.

The heating of both the tissue and bioprobes increase with increased AMF amplitude. At low AMF amplitudes, small increases yield significant increases in magnetic heating. As the bioprobes approach saturation, however, their relationship with the AMF amplitude becomes one of diminishing return. This relationship is unique to the particular magnetic material, as are the values that constitute “low” or “saturating” AMF amplitudes. Bioprobe heating is at first related to the AMF amplitude by an exponent greater than one (1), which gradually diminishes to an exponent less than one (1) as saturation is approached. At typical pulse widths and duty cycles, eddy current heating is directly related to duty cycle. The capability to pulse the generator output allows the benefits of operating at higher AMF amplitudes while maintaining a constant reduced tissue heating by reducing the duty cycle.

It is desirable to apply the AMF to treatment area 205 of the subject. Generating high peak amplitude AMF over a large area requires a very large AMF generator and exposes large amounts of healthy tissue to unnecessary eddy current heating. Without some way of directing the field to where it is useful, disease in the chest or trunk may only be practically treated by placing the patient within a large solenoid coil. This would expose most of the major organs to eddy current heating, which must then be monitored and the AMF adjusted so as not to overheat any part of a variety of tissue types. Each of these tissue types has a different rate of eddy current heating. The peak AMF strength would need to be reduced to protect those tissue types that experience the most extreme eddy current heating. If the varieties of exposed tissue are minimized, it is likely that the AMF strength can be increased, thereby reducing the treatment time and increasing the efficacy. One method of confining the high peak amplitude AMF to treatment area 205 is by defining the lowest reluctance path of magnetic flux with high permeability magnetic material. This path is referred to as a magnetic circuit (102 and 602). The magnetic circuit may be provided so that all or most of the magnetic flux produced by solenoid coil 622 (FIG. 6) may be directed to the treatment area 205. One benefit of magnetic circuit 602 is that the necessary amount of flux may be reduced since the amount of flux extending beyond treatment area 205 is minimized. Reducing the required flux reduces the required size and power of the AMF generator, and minimizes exposure of tissue outside treatment area 205 to high peak amplitude AMF. In addition, a reduced area of AMF exposure avoids the unintentional heating of surgical or dental implants and reduces the likelihood that they will need to be removed prior to treatment, thereby avoiding invasive medical procedures. Concentrating the field permits the treatment of large volumes within the chest or trunk with a portable size device.

The material used to fabricate magnetic circuit 602 may be appropriate to the peak amplitude and frequency of the AMF. The material may be, but is not limited to, iron, powdered iron, assorted magnetic alloys in solid or laminated configurations and ferrites. Pole faces 104, 204, and 604 may be shaped and sized to further concentrate the flux produced in the treatment area.) Different pole pieces having different sizes and shapes may be used, so that the treatment area and volume may be adjusted. When passing from one material to another, lines of magnetic flux 612 travel in a direction normal to the plane of the interface plane. Thus, face 604 may be shaped to influence the flux path through gap 633. Pole faces 604 may be detachable and may be chosen to extend the magnetic circuit 602 as much as possible, to minimize gap 633 while leaving sufficient space to receive that portion of the patient being treated. The addition of secondary coils can aid in the concentration of the field as well as reducing the field strength in sensitive areas.

The magnetic field will be most intense close to coil 622 and will diminish exponentially as the distance from the coil increases. This characteristic provides for high field strength in the tissue near the surface while minimizing the exposure of deeper tissues.

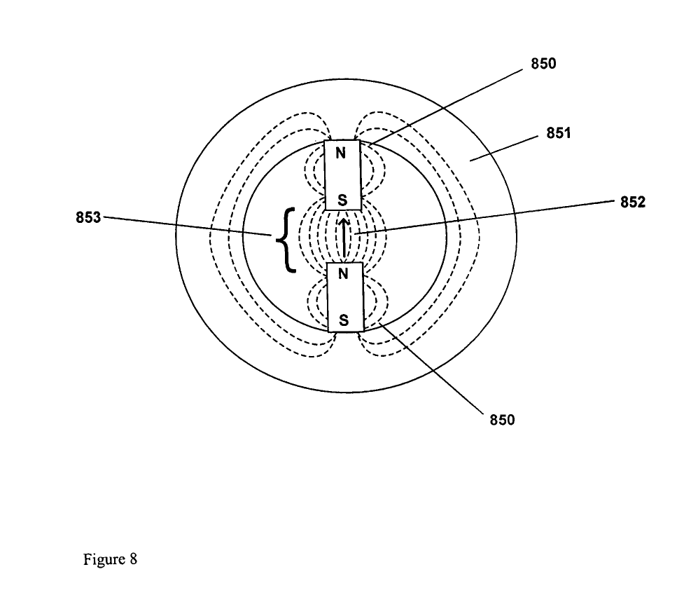

An alternative device for producing AMF, as depicted in the embodiment in FIG. 8, features a circular shaped rotor 851 comprising a magnetic material or magnets 850, which provides a low magnetic reluctance return path. Magnets 850 may be attached to or mounted on rotor 851. Magnets 850 and rotor 851 are spun around a targeted treatment area 852. Magnets 850 are shaped such that the return path between poles of a single magnet 850 is of higher reluctance than the return path comprising a gap 853 and rotor 851. As rotor 851 turns, the net magnetic field in gap 853 is of constant amplitude with an angular velocity equal to the rotational velocity of rotor 851. A stationary ferro or ferrimagnetic target located within gap 853 would experience hysteretic heating as well as eddy current heating. The eddy current heating of targeted treatment area 852 could differ from that due to traditional AMF on a fixed axis, and would depend upon the shape of targeted area 852, the orientation of the body comprising targeted area 852 relative to rotor 851, and the distribution of resistivity within targeted body in the targeted area.

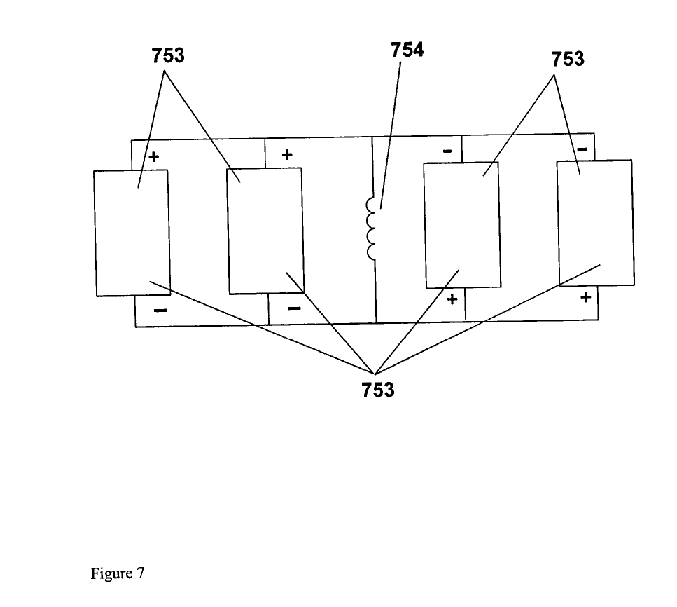

Another alternative device comprises a pair or pairs of pulse modulators 753 similar to those used in pulsed radar transmitters, as illustrated in FIG. 7. Either line type or hard tube modulators may be used. Modulators 753 are coupled to an inductor 754 in pairs with opposite polarity (753′ and 753″) and diode protected. High power modulators of this type have been designed to operate at several kilohertz. They fire alternately, causing both positive and negative current through the inductor. The maximum frequency of each pulse-forming element is limited by the charging time of the energy storage device (e.g., storage capacitor or pulse forming network (PFN)), or by the recovery time of the switch (e.g., IGBT, hydrogen thyratron, SCR, MOSFET, or spark gap). For higher frequencies, multiple pairs may be employed and fired sequentially.

2.5. The Inductor for the Targeted Thermotherapy System

An inductor is used for inductively heating the bioprobes. The inductor can be a C-shaped or M-shaped high magnetic-flux material. The inductor can be a single-turn coil or a multi-turn coil. The coil may be coated with an appropriate insulating material for placement directly on the skin of a patient.

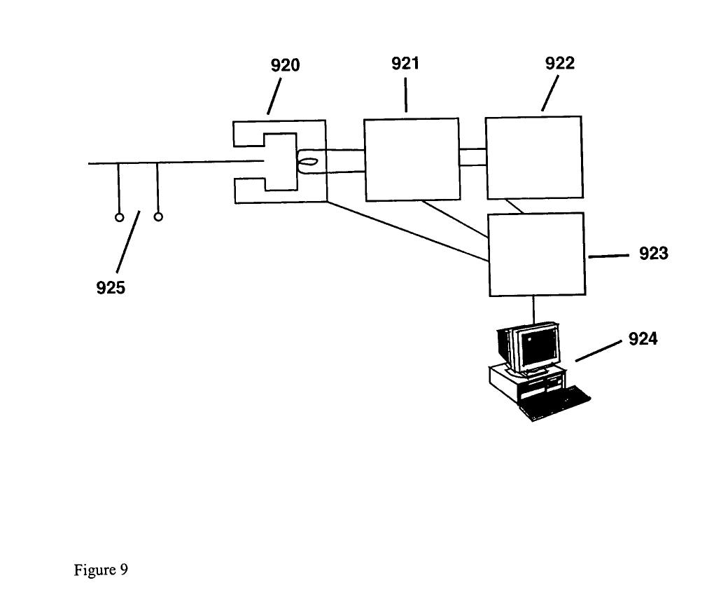

FIG. 9 is a block diagram illustrating one embodiment of the targeted thermotherapy system. The portion of the subject to be treated is prepared for exposure to an AMF by positioning it in an inductor 920 via a subject interface 925, which can be, for example, a bed or a seat. The system comprises a tank circuit 921 that matches the impedance between a generator 922 and inductor 920. The operator controls the procedure via a controlling unit 923 using a console 924.

The induction process is carried out at a frequency range of from about 50 Hz to about 2 MHz, preferably from about 100 kHz to about 500 kHz, and more preferably at about 150 kHz.

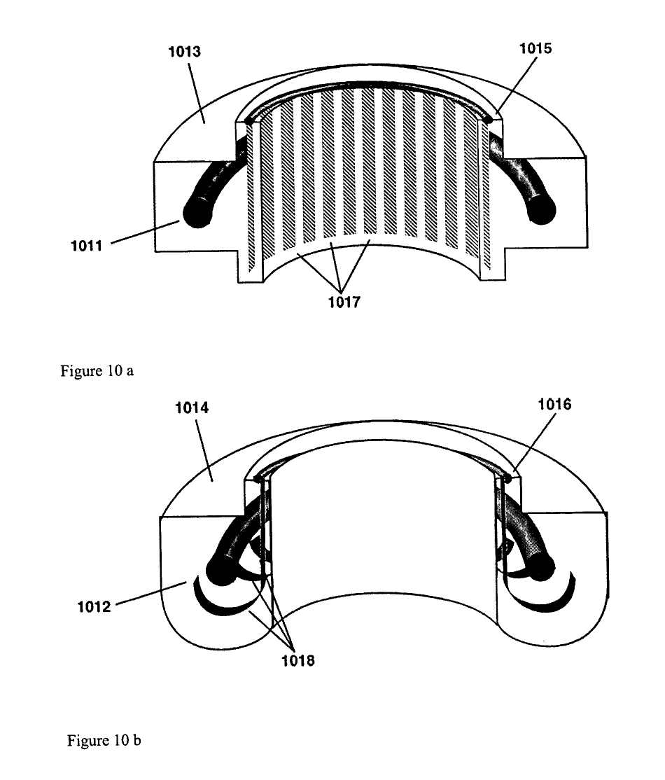

In one embodiment of the invention, the inductor is a single-turn coil. Two examples of coil arrangements that eliminate the electrical component of the RF field are illustrated in FIGS. 10a and 10b. FIG. 10a illustrates an arrangement in which the subject is located within an inductor coil 1011, where inductor coil 1011 surrounds the subject. FIG. 10b illustrates an inductor coil 1012, which is placed, e.g., dorsal or anterior to the subject. The subject is located proximal to that side of the arrangement, as illustrated in FIG. 10b, where the shielding metal plates 1018 bend. These shielding plates shield the subject's body from the electrical component of the RF radiation, which itself might heat up the tissue. Inductor coils 1011 and 1012 are constructed from a tube through which water flows to cool the inductor coil. The tubing material can be any suitable material, such as copper, so as to better facilitate heat conduction.

Metal plates 1017 and 1018 are formed as stripes, and are located in coil arrangements in such a way that they are in parallel to the field lines of the magnetic RF component, and perpendicular to the field lines of the electrical component. This arrangement results in a passage of the magnetic field lines and a blockage of the electrical field lines of the RF field. These metal plates can be fabricated from any suitable material, such as copper, for better heat conduction. A cooling tube 1015 or 1016 is attached to metal plates 1017 or 1018. The coil arrangement is covered with an electrical insulating cover 1013 or 1014, which may be fabricated from any suitable plastic, such as polytetrafluorethylene (PTFE), polyetheretherketone (PEEK), polyester (PE), polypropylene (PP) or polyurethane (PU).

Metal plates 1017 or 1018 typically are about 1 mm to about 4 mm wide and about 0.2 mm to about 0.5 mm thick. The water flows through inductor coil 1011 or 1012 preferably at a rate of about 4 liter/minute to about 20 l/min at 1 bar to 10 bar.

One of the most important and constantly growing imaging modalities in radiology is magnetic resonance imaging (MRI). For spatial encoding and image reconstruction gradient magnetic fields are superposed onto the static main magnetic field (Bo). Gradient coils can be applied in three independent spatial directions (x,y,z). While state of the art MRI machines have magnetic flux densities of 3 Tesla (30,000 Oersted), developments are under way to the 8 Tesla technology. On the market are 3 Tesla machines with 40 millitesla per meter (400 Oerstedt/meter) gradient fields. A 7 Tesla machine with 250 millitesla per meter (2,500 Oerstedt per meter) gradient field is in development stages. In one embodiment of the invention, the bioprobes in the subject are heated using the switching of the gradient coils of an MRI.

The repetition time T_{R} of the MRI determines the frequency of the gradient coil inducing AMF. At present, T_{R}=100 μsec. (f_{AMF}=10 kHz) seems to be an upper limit. However, one could sequentially switch the three independent special gradients x, y, and z to create a three times higher frequency. A further advantage of this technology would be the generation of a rotating magnetic field.

It is believed that future MRI technology will use higher gradient field strength and faster gradient coil switching.

3. Inhibiting or Destroying the Vascularity of the Tumor (Devascularization)

The heat generated using the targeted therapy approach induces thrombosis and necrosis in the overall tumor tissue area and destroys the vasculature of the tumor. Although not limited by theory, it is believed that the therapeutic effect of the targeted therapy approach is better than those therapies listed in Table III (below) due to the combination of antibody-targeted cell killing by necrosis and apoptosis as well as inactivating the vascularity which results in the inhibition of the blood supply of the tumor.

This combination effect could be combined with therapies that target the vascularity of the tumor tissue. There are various tumor cell targeted and non-targeted approaches of inactivating the vasculature of solid tumors (see e.g., U.S. Pat. Nos. 5,855,866, 6,051,230, 6,093,399, 6,004,555, and U.S. Patent Application No. US2003/0129193). Those therapies enhance the coagulant status of the vasculature utilizing a sensitising agent and/or utilize a tumor-targeted coagulant effective to induce coagulation in the vasculature of the tumor.

The sensitizing agent may be an endotoxin or a detoxified endotoxin derivative. The sensitizing agent can be monophosphoryl lipid A (MPL), monocyte chemoattractant protein-1 (MCP-1), platelet-derived growth factor-BB (PDGF-BB), C-reactive protein (CRP), tumor necrosis factor-α (TNF-α) or inducer of TNF-α, a Rac1 antagonist, DMXAA, CM101 or thalidomide, muramyl dipeptide (MDP), threonyl-MDP or MTPPE, anti-angiogenic agent, vasculostatin, canstatin or maspin, VEGF inhibitor, anti-VEGF blocking antibody, VEGF receptor construct (sVEGF-R), tyrosine kinase inhibitor, antisense VEGF construct, anti-VEGF RNA aptamer, anti-VEGF ribozyme, antibody that binds to the cell surface activating antigen CD40, sCD40-Ligand (sCD153), combretastatin A-1, A-2, A-3, A-4, A-5, A-6, B-1, B-2, B-3, B-4, D-1 or D-2, thalidomide, or any combination thereof.

The binding region of the tumor-targeted coagulant can be an antibody, antigen-binding region, monoclonal, recombinant, human, or part-human, or humanized antibody, chimeric antibody, scFv, Fv, Fab′, Fab, diabody, F(ab′)₂, ligand, VEGF receptor, an FGF receptor, a TGF-β receptor, TIE, VCAM-1, ICAM-1, P-selectin, E-selectin, PSMA, pleiotropin, endosialin, endoglin, fibronectin, scatter factor/hepatocyte growth factor (HGF), platelet factor 4 (PF4), PDGF, or TIMP.

In one embodiment of the invention, targeted therapy is combined with an agent sensitizing the coagulant status of the tumor, where the sensitizing agent may be administered prior to, during, or after the targeted therapy administration.

In another embodiment of the invention, the bioprobes are retained within both the tumor vasculature and the walls of individual tumor cells by the presence of the chemical marker to which the tumor-targeted coagulant is specific. The bioprobes can then be exposed to the AMF. The heat generated by the bioprobes serve to destroy or disrupt the tumor vasculature, in addition to the individual cell walls.

In another embodiment of the invention, the targeted therapy is combined with a tumor targeted coagulant agent effective to induce coagulation in the vasculature of the tumor, where the coagulant agent may be administered prior to, during, or after the targeted therapy administration, or any combination thereof. A combination of an agent sensitizing the coagulant status of the tumor and a tumor targeted coagulant agent may also be used with targeted therapy.

The past few years have been difficult for companies developing pharmaceuticals that fight cancer by attacking the blood vessels that feed tumors (antiangiogenesis). These antiangiogenesis drugs produced some small benefits in early clinical trials; however, such benefits were attained at the expense of undesirable side effects. Pharmaceuticals involving antiangiogenesis, that are currently under development, are listed in Table III. In one embodiment of the invention, the targeted therapy system is used in combination with at least one of these pharmaceuticals, or similar pharmaceuticals that will be developed in the future.

TABLE III
PHARMECEUTICALS INVOLVING ANTIANGIOGENESIS

| Drug | Company | Action(s) |
|---|---|---|
| Bevacizumab, | Genentech | Blocks VEGF activity |
| RhuMAb-VEGF | | |
| (Also known as | | |
| Avastin ™) | | |
| BMS-275291 | Bristol-Myers Squibb | Metalloproteinase |
| | | inhibitor |
| Celecoxib | Pharmacia/Pfizer | Inhibits angiogenic |
| | | growth factor production |
| EMD121974 | Merck KGaA | Integrin inhibitor |
| rhEndostatin | EntreMed | Integrin inhibitor; |
| (also known as | | other actions |
| Endostatin) | | |
| Cetuximab (also | ImClone Systems | Inhibits EGF receptor |
| known as | | |
| Erbitux) | | |
| Interferon-α | Hoffmann-La Roche | Inhibits FGF production |
| LY317615 | Eli Lilly | Protein kinase C inhibitor |
| AE-941 | Aeterna Laboratories | Inhibits VEGF |
| (also known as | | and metalloproteinases; |
| Neovastat) | | promotes apoptosis |
| PTK787 | Abbott Laboratories | Inhibits VEGF and |
| | | other receptors |
| SU6668 | Sugen | Blocks VEGF and |
| | | PDGF receptors |
| SU11248 | Sugen | Blocks VEGF, PDGF, |
| | | and other receptors |
| Thalidomide | Celgene Corp. | Unknown |
| VEGF-Trap | Regeneron | Blocks VEGF activity |
| | Pharmaceuticals | |
| ZD1839 (Iressa) | AstraZeneca | Blocks EGF receptor |
| ZD6474 | AstraZeneca | Blocks VEGF and |
| | | EGF receptors |

4. Combination Therapies

Targeted thermotherapy may be applied in combination with other therapies to enhance the therapeutic effect. For example, targeted thermotherapy may be combined with hyperthermia, direct antibody therapy, radiation therapy, chemo- or pharmaceutical therapy, surgical or interventional techniques, bone marrow and stem cell transplantation, or any combination thereof.

4.1. Targeted Thermotherapy in Combination with Hyperthermia

Energy can generate heat within the human body by different mechanisms. Local hyperthermia is beneficial to enhance the targeted therapeutic system, preferably in the temperature range from about 38° C. to about 48° C., more preferably from about 42° C. to about 45° C. for the duration of the treatment with targeted therapy or longer. In one embodiment of the invention, hyperthermia is administered at least once prior to, during, or at least once after the completion of the targeted therapy administration, or any combination thereof. Typically, the hyperthermia treatment is administered for a period of time from about 30 seconds to about 30 minutes, preferably from about 30 seconds to about 3 minutes.

Eddy currents are induced in and around conductive tissue parts or body parts that contain conductive material, such as the bowel, intestine or stomach, when placed in AMF. Eddy currents can be used to generate hyperthermia in the tissue in combination with targeted bioprobes to enhance the therapeutic effect of the targeted thermotherapy. In one embodiment of the present invention, the eddy currents are locally enhanced by local injection of conductive substances, such as NaCl solution. In another embodiment, eddy currents in the gastrointestinal body parts are enhanced with the administration of conductive nutrition to the patient prior to the targeted therapy administration. Eddy currents in the gastrointestinal body parts may be reduced with the administration of enema prior to targeted therapy administration.

Light can be used as an energy source for hyperthermia in combination with the targeted thermotherapy. Light energy source can be applied locally in small areas or radiated onto larger body parts. Light energy source can also be applied by non-magnetic and non-conductive glass fibers through plastic endoscopes, catheters or plastic or ceramic needles, or by non-magnetic and non-conductive glass rods through plastic endoscopes, catheters, or plastic or ceramic needles when used during targeted therapy administration.

RF and microwave radiation can also be used to produce hyperthermia in combination with targeted thermotherapy. The frequency of the RF or microwave for the additional treatment is different from the frequency for targeted thermotherapy. Electromagnetic radiation in the range above 900 kHz will be absorbed directly from the tissue. Frequencies below 900 kHz will cause eddy current heating.

Alternating or direct currents flowing though the body can be used to produce hyperthermia in combination with the targeted thermotherapy. These currents can be applied locally by deploying two electrodes near the tissue targeted for heating on opposite sides outside the main targeted therapy AMF region, also referred to as bipolar currents. These currents can also be applied by placing one electrode at a location far from the AMF and one electrode variable near the targeted treatment location, also referred to as monopolar currents.

Thermal seeds are metallic implants that are deployed temporarily or permanently in tissue targeted for heating, and heated inductively. These thermal seeds can be used in combination with the targeted nano therapy; the same AMF is used to heat these seeds, however a different superposed AMF of different field strength and/or frequency can also be used. Thermal seeds can comprise metal alloys such as PdCo, FeNi, stainless steel or titanium alloys. These seeds can be coated with a conductive material that is more electrically conductive than PdCo, FeNi, stainless steel or titanium alloys, such as gold, to enhance the eddy currents induced in the outer layer of the seeds. Thermal seeds may further comprise a biocompatible coating, thermal conductive coating, or a combination thereof.

In one embodiment of the invention, thermal baths of hot or warm water, oils or other solutions is used to generate hyperthermia.

In another embodiment of the invention, non-targeted particle heating is used in combination with targeted thermotherapy. Bioprobes with or without antibodies are injected directly into the tissue targeted for treatment and heated with AMF.

In another embodiment of the invention, hyperthermia is generated by induction of non-targeted bioprobes.

In yet another embodiment of the invention, ionizing radiation is used to produce hyperthermia, which is than used in combination with targeted thermotherapy. The ionizing radiation source can be alpha particles, beta particles, gamma particles, or any other high-energy particle, or x-ray or gamma radiation.

4.2. Targeted Thermotherapy in Combination with Direct Antibody Therapy

Monoclonal antibodies (MAB's) work on disease cells such as cancer cells in the same way natural antibodies work, by identifying and binding to the target cells. They then alert other cells in the immune system to the presence of the cancer cells. MAB's are specific for a particular antigen. MAB's are classified as Biological Response Modifiers. Because MAB's affect the immune system, their use is referred to as immunotherapy rather than chemotherapy, which utilize pharmaceuticals that interfere with cancer cell growth. MAB's by themselves may enhance a patient's immune response to the cancer. Efficacy has been seen in clinical trials that utilize antibodies targeting tumor cell surface antigens such as B-cell idiotypes, CD20 on malignant B cells, CD33 on leukemic blasts, and HER2/neu on breast cancer. (see e.g., Weiner LM., Monoclonal Antibody Therapy of Cancer, Semin. Oncol. 1999 Oct.; 26 (5 Suppl 14):43-51). In one embodiment of the invention, MAB therapy is administered at least once prior to, or at least partly during, or at least once after targeted therapy administration, or any combination thereof.

4.3. Targeted Thermotherapy in Combination with Radiation Therapy

Radiotherapy, also referred to as radiation therapy, is the treatment of cancer and other diseases utilizing ionizing radiation. Ionizing radiation deposits energy that injures or destroys cells in the area being treated (the “target tissue”) by damaging their genetic material, making it impossible for these cells to continue to grow. Although radiation damages both cancer cells and normal cells, the latter are able to repair themselves and function properly. Radiotherapy may be used to treat localized solid tumors, such as cancers of the skin, tongue, larynx, brain, breast, or uterine cervix. It can also be used to treat leukemia and lymphoma (cancers of the blood-forming cells and lymphatic system, respectively). In one embodiment of the present invention, radiotherapy or radiation therapy is used in combination with targeted thermotherapy. Radiotherapy is applied at least once prior to, or at least partly during, or at least once after targeted therapy administration, or any combination thereof.

One type of radiation therapy commonly used involves x-rays or gamma rays. X-rays were the first form of photon radiation to be used to treat cancer. Depending on the amount of energy they possess, the rays can be used to destroy cancer cells on the surface of or deeper in the body. The higher the energy of the x-ray beam, the deeper the penetration of the x-rays into the target tissue. Linear accelerators and betatrons are machines that produce x-rays of increasingly greater energy. The use of machines to focus radiation (such as x-rays) on a cancer site is referred to as external beam radiotherapy. These beams are shielded from the outside world and special shielding is used for “focusing” these beams onto defined body areas. In one embodiment of the invention, external beam radiotherapy is used in combination with targeted thermotherapy. If both the targeted thermotherapy and radiotherapy methods are used simultaneously, the AMF system may comprise a separate opening for the beam to enter. Alternatively, the beam may be directed through the patient's opening (patient gantry). Intraoperative irradiation is a technique in which a large dose of external radiation is directed at the tumor and surrounding tissue during surgery.

Gamma rays are produced spontaneously as certain elements (such as radium, uranium, and cobalt 60) release radiation as they decompose or decay. Each element decays at a specific rate and emits energy in the form of gamma rays and other particles. X-rays and gamma rays have the same effect on cancer cells.

Another investigational approach is particle beam radiation therapy. This type of therapy differs from photon radiotherapy as it uses fast-moving subatomic particles to treat localized cancers. Particle accelerators are used to produce and accelerate the particles required for this procedure. Some particles (neutrons, pions, and heavy ions) deposit more energy than x-rays or gamma rays along the path they take through tissue, thus causing more damage to the cells they contact. This type of radiation is often referred to as high linear energy transfer (high LET) radiation. In one embodiment of the invention, high LET therapy is used in combination with targeted thermotherapy.

Another technique for delivering radiation to cancer cells is to place radioactive implants directly in a tumor or in a body cavity. This is referred to as internal radiotherapy. (Brachytherapy, interstitial irradiation, and intracavitary irradiation are types of internal radiotherapy.) During this treatment, the radiation dose is concentrated in a small area, and the procedure may require the patient to stay in the hospital for a few days. In one embodiment of the invention, internal radiotherapy is used in combination with targeted thermotherapy. The implant comprises a material that heats during the targeted therapy administration by eddy current or hysteretic heating, or comprises a material that does not heat under AMF exposure, such as plastic, ceramic, glass, or transplanted human tissue.

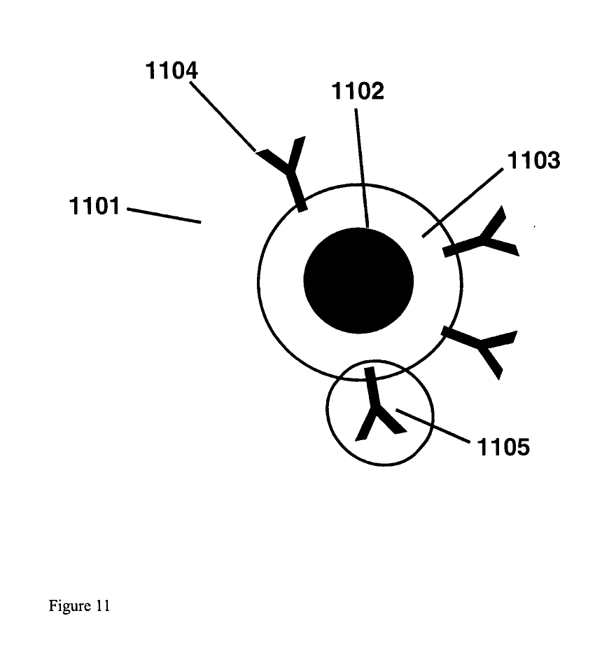

In one embodiment of the invention, radiolabled antibodies deliver doses of radiation directly to the cancer site (radioimmunotherapy) in combination with targeted thermotherapy. FIG. 11 illustrates a bioprobe 1101, which is attached to at least one radioisotope 1105. Such a bioprobe can be a dual therapy bioprobe. Once injected into the body, the antibodies actively seek out the cancer cells, which are destroyed by the cell-killing (cytotoxic) action of the radiation.

Examples of radioisotopes suitable for use herein are:
Molybdenum-99: Used as the ‘parent’ in a generator to produce technetium-99 m, the most widely used isotope in nuclear medicine.
Technetium-99 m: Used particularly for imaging the skeleton and heart muscle, and for imaging the brain, thyroid, lungs (perfusion and ventilation), liver, spleen, kidney (structure and filtration rate), gall bladder, bone marrow, salivary and lacrimal glands, heart blood pool, infection and numerous specialized medical studies.
Chromium-51: Used for labeling red blood cells and quantifying gastro-intestinal protein loss.
Cobalt-60: Used for external beam radiotherapy.
Copper-64: Used for studying genetic diseases affecting copper metabolism, such as Wilson's and Menke's diseases.
Dysprosium-165: Used as an aggregated hydroxide for synovectomy treatment of arthritis.
Ytterbium-169: Used for cerebrospinal fluid studies in the brain.
Iodine-125: Used in cancer brachytherapy (prostate and brain), also used for diagnostic evaluation of the kidney filtration rate and for diagnosing deep vein thrombosis in the leg. It is also widely used in radioimmuno assays to show the presence of hormones in small quantities.
Iodine-131: Widely used in treating thyroid cancer and in imaging the thyroid; also used in the diagnosis of abnormal liver function, renal (kidney) blood flow and urinary tract obstruction. Although it is a strong gamma emitter, it is used for beta therapy.
Iridium-192: Supplied in wire form for use as an internal radiotherapy source for cancer treatment.
Iron-59: Used for studying iron metabolism in the spleen.
Phosphorus-32: Used in the treatment of polycythemia vera (excess red blood cells). It is a beta emitter.
Potassium-42: Used for the determination of exchangeable potassium in coronary blood flow.
Rhenium-188 (derived from Tungsten-188): Used for beta irradiating coronary arteries from an angioplasty balloon.
Samarium-153: Very effective in relieving the pain of secondary cancers lodged in the bone. It is commercially available as Quadramet™. Also, it is very effective for prostate and breast cancer. It is a beta emitter.
Selenium-75: Used in the form of seleno-methionine to study the production of digestive enzymes.
Sodium-24: Used for studies of electrolytes within the body.
Strontium-89: Very effective in reducing the pain of prostate cancer. Beta emitter.
Xenon-133, Xenon-127: Used for pulmonary (lung) ventilation studies.
Yttrium-90: Used for cancer therapy and as silicate colloid for the treatment of arthritis in larger joints. It is a beta emitter.

Radiation therapy in combination with targeted thermotherapy may be used alone or in combination with chemotherapy, surgery or both.

4.4. Targeted Thermotherapy in Combination with Chemo- or Pharmaceutical Therapy

Chemotherapy is the treatment of diseases, such as cancer, with drug therapy. For most types of cancer, chemotherapy often requires the use of a number of different drugs or agents; this is referred to as combination chemotherapy. Chemotherapy may be administered in a variety of ways, such as intravenously (IV; into a vein is the most common), intramuscularly (IM; injection into a muscle), orally (by mouth), subcutaneously (SC; injection under the skin), nitralesionally (IL; directly into a cancerous area), intrathecally (IT; into the fluid around the spine), or topically (application onto the skin). Tumor cell resistance to various chemotherapeutic agents represents a major problem in clinical oncology. Thus, many of the most prevalent forms of human cancer still resist effective chemotherapeutic intervention, despite the many advances in the chemotherapy of neoplastic disease during the last 30 years.

The cell cycle is composed of four phases during which the cell prepares for and effects mitosis. Cells that are committed to divide again enter the G₁ phase. Preliminary synthetic cellular processes that occur prepare the cell to enter the DNA synthetic (S) phase. Specific protein signals regulate the cell cycle and allow replication of the genome where the DNA content becomes tetraploid (4N). After completion of the S phase, the cell enters a second resting phase, G₂, prior to undergoing mitosis. The cell progresses to the mitotic (M) phase, in which the chromosomes condense and separate and the cell divides, producing two daughter cells. Chemotherapeutic agents used in combination with targeted thermotherapy can be classified according to the phase of the cell cycle in which they are active.
S phase-dependent agents: Antimetabolics (Capercitabine, Cytarabine, Doxorubicin, Fludarabine, Floxuridine, Fluorouracil, Gemcitabine, Hydroxyurea, Mercaptopurine, Methotrexate, Prednisone, Procarbazine, and Thioguanine).
M phase-dependent agents: Vinca alkaloids (Vinblastine, Vincristine, and Vinorelbine), Podophyllotoxins (Etoposide, and Teniposide), Taxanes (Doxetaxel), and Paxlitaxel.
G₂ pase-dependent agents: (Bleomycin, Irinotecan, Mitoxantrone, and Topotecan).
G₁ pase-dependent agents: (Asparaginase, and Corticosteroids). Chemotherapeutic drugs, as classified by mechanism of action, that can be combined with the targeted thermotherapeutic system are:
Alkylating agents that impair cell function.
Nitrogen mustards, which are powerful local vesicants, such as (mechlorethamine (Mustargen), cyclophosphamide, ifosfamide (Ifex), and chlorambucil (Leukeran)).
Nitrosoureas, which are distinguished by their high lipid solubility and chemical instability, rapidly and spontaneously decompose into two highly reactive intermediates: chloroethyl diazohydroxide and isocyanate. The lipophilic nature of the nitrosoureas enables free passage across membranes; therefore, they rapidly penetrate the blood-brain barrier, achieving effective CNS concentrations. Accordingly, these agents are used for the treatment of a variety of brain tumors.
Platinum agents include Cisplatin (Platinol) and Carboplatin (Paraplatin).
Antimetabolites are structural analogs of the naturally occurring metabolites involved in DNA and RNA synthesis. As the constituents of these metabolic pathways have been elucidated, a large number of structurally similar drugs have been developed that alter the critical pathways of nucleotide synthesis.
Antimetabolites exert their cytotoxic activity either by competing with normal metabolites for the catalytic or regulatory site of a key enzyme, or by substituting for a metabolite that is normally incorporated into DNA and RNA. Because of this mechanism of action, antimetabolites are most active when cells are in S phase and have little effect on cells in GO. Consequently, these drugs are most effective in tumors that have a high growth fraction.
Natural products are compounds possessing antitumor activity that have been isolated from natural substances, such as plants, fungi, and bacteria.
Antitumor antibiotics, particularly Bleomycin (Blenoxane), preferentially intercalate DNA at guanine-cytosine and guanine-thymine sequences, resulting in spontaneous oxidation and formation of free oxygen radicals that cause strand breakage.
Anthracyclines.
Epipodophyllotoxins, particularly Etoposide (VP-16 [VePesid and others]), are semisynthetic epipodophyllotoxin extracted from the root of Podophyllum peltatum (mandrake). Epipodophyllotoxins inhibit topoisomerase II activity by stabilizing the DNA-topoisomerase II complex; this ultimately results in the inability to synthesize DNA, and the cell cycle is stopped in G₁ phase.
Vinca alkaloids are derived from the periwinkle plant, Vinca rosea. Upon entering the cell, vinca alkaloids bind rapidly to the tubulin. The binding occurs in S phase at a site different from that associated with paclitaxel and colchicine. Thus, polymerization of microtubules is blocked, resulting in impaired mitotic spindle formation in the M phase.
Taxanes, particularly Paclitaxel (Taxol) and docetaxel (Taxotere), are semisynthetic derivatives of extracted precursors from the needles of yew plants. These drugs have a novel 14-member ring, the taxane. Unlike the vinca alkaloids, which cause microtubule disassembly, the taxanes promote microtubule assembly and stability, therefore blocking the cell cycle in mitosis. Docetaxel is more potent in enhancing microtubule assembly and also induces apoptosis.
Camptothecin analogs are semisynthetic analogs of the alkaloid camptothecin, (derived from the Chinese ornamental tree, Camptotheca acuminata) that inhibit topoisomerase I and interrupt the elongation phase of DNA replication.

In one embodiment of the present invention, the targeted thermotherapeutic system is utilized in combination with chemotherapy. Chemotherapy can be administered at least once prior to, or at least partly during, or at least once after the targeted therapy administration, or any combination thereof.

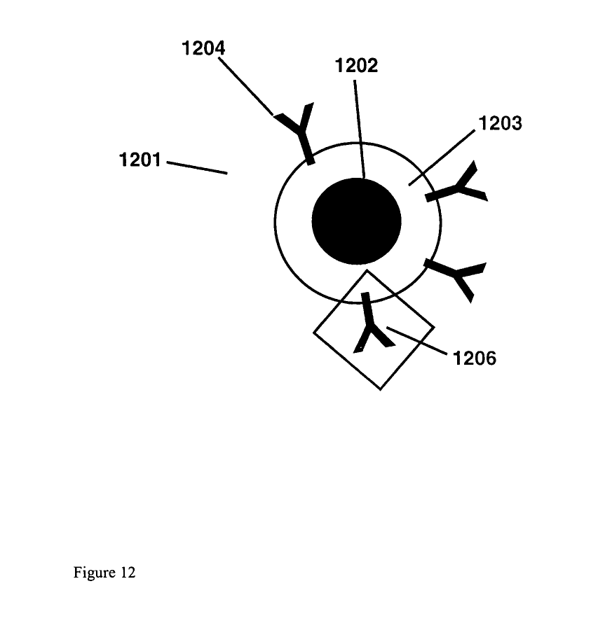

The chemotherapeutic drug or agent may also be attached to the bioprobe. FIG. 12 illustrates a configuration comprising bioprobe 1201, which is attached to a chemotherapeutic drug or agent 1206. Such a bioprobe would constitute a dual therapy bioprobe. The drug or agent can be a S phase-dependent antimetabolics, capercitabine, cytarabine, doxorubicin, fludarabine, floxuridine, fluorouracil, gemcitabine, hydroxyurea, mercaptopurine, methotrexate, prednisone, procarbazine, thioguanine, M phase-dependent vinca alkaloids, vinblastine, vincristine, vinorelbine, podophyllotoxins, etoposide, teniposide, taxanes, doxetaxel, paxlitaxel, G₂ pase-dependent, bleomycin, irinotecan, mitoxantrone, topotecan, G₁ pase-dependent, asparaginase, corticosteroids, alkylating agents, nitrogen mustards, mechlorethamine, mustargen, cyclophosphamide, ifosfamide (Ifex), and chlorambucil, leukeran, nitrosoureas, platinum agents, cisplatin, platinol, carboplatin, paraplatin, antimetabolites, natural therapeutic products, antitumor antibiotics, bleomycin, anthracyclines, epipodophyllotoxins, vinca alkaloids, taxanes, camptothecin, or any combination thereof.

Monoclonal antibodies (MAB's) can be bound to a chemotherapy agent. This combination allows for two mechanisms of attacking the cell: 1) the chemical from the chemotherapy, and 2) the immune response from the MAB. Chemotherapy can be more effective when the cells are weakened by the MAB.

In one embodiment of the invention, targeted thermotherapy is combined with chemotherapeutic drugs or agents attached to MAB's. These agents can be administered prior to, during, or after targeted therapy administration. In another embodiment, the chemotherapeutic drug or agent is activated during the AMF exposure as it is released from the bioprobe due to the inductive heating. The drug or agent can also be destroyed when the AMF is turned on. In an alternative embodiment, the drug or agent is incorporated into coating 1203 and released when the AMF is turned on. Coating 1203 may comprise one or more layers, where the layers may be of the same or different material, and the drug or agent may be incorporated into one or more of the coating layers.

Most traditional approaches to cancer therapy attempt to destroy individual cancer cells. Drugs that target cancer cells must overcome a significant number of structural barriers within the tumor in order to be effective. They must first exit the tumor blood vessels, migrate past the support structures that underlie the vessels and eventually make their way to the cancer cells. As result of these structural barriers, very little drug injected into the blood stream of a patient is able to reach and destroy cancer cells. One potential solution to this problem is to increase the permeability of the blood vessels within the tumor, which will permit more therapeutic drug to reach and kill substantially more cancer cells. Vasopermeation Enhancement Agents (VEA's) are a new class of drugs designed to increase the uptake of cancer therapeutics and imaging agents at the tumor site, potentially resulting in greater efficacy. VEA's work by using monoclonal antibodies, or other biologically active targeting agents, to deliver known vasoactive compounds (i.e., molecules that cause tissues to become more permeable) selectively to solid tumors. Once localized at the tumor site, VEA's alter the physiology and the permeability of the vessels and capillaries that supply the tumor. In pre-clinical studies, drug uptake has been increased up to 400% in solid tumors when VEA's were administered several hours prior to the therapeutic treatment. VEA's are intended for use as a pre-treatment for most existing cancer therapies and imaging agents. VEA's may be effective across multiple tumor types. Examples of VEA's include the commercially available Cotara™ and Oncolym® (Peregrine Pharmaceuticals, Inc., Tustin, Calif.). VEA's can be used with the targeted thermotherapeutic therapy to enhance the blood flow and hence the uptake of bioprobes at the tumor cells.

4.5. Targeted Thermotherapy in Combination with Surgical or Interventional Techniques

In one embodiment of the invention, targeted thermotherapy is combined with open or minimally invasive surgery or with other interventional techniques. During the operation or the intervention, the bioprobes can be heated with the AMF. The AMF energy source may be a part of the operational space and thus covered in sterile material. In such instances, all surgical tools are made from non-magnetic materials such as plastic, ceramic, glass or non-magnetic metals or metal-alloys (titan). The AMF energy source may be located next to the sterile surgical site, and the patient can be moved in and out the AMF energy field, in a manual or automatic manner.

In one embodiment of the invention, an organ is surgically prepared to be lifted to outside the patient's body, while it continues to be anatomically and physiologically attached to the body, and irradiated with the AMF extracorporeally. The treated organ is then replaced into the patient's body. Such a technique allows for enhanced selectivity of the AMF to only the targeted organ, while other parts of the body are unexposed to the AMF.

Targeted therapy can be administered at least once prior to, at least partly during, at least once after surgery or other interventional technique, or any combination thereof.

4.6. Targeted Nano Therapy in Combination of Bone Marrow and Stem Cell Transplantation

Bone marrow contains immature cells referred to as stem cells that produce blood cells. Most stem cells are found in the bone marrow, but some stem cells referred to as peripheral blood stem cells (PBSC's) can be found in the bloodstream. Stem cells can divide to form more stem cells, or they can mature into white blood cells, red blood cells, or platelets.

Bone marrow transplantation (BMT) and peripheral blood stem cell transplantation (PBSCT) are procedures that restore stem cells that have been destroyed by high doses of chemotherapy and/or radiation therapy.

The primary purpose of BMT and PBSCT in cancer treatment is to make it possible for patients to receive very high doses of chemotherapy and/or radiation therapy. Without healthy bone marrow, the patient is no longer able to make the blood cells needed to carry oxygen, defend against infection, and prevent bleeding. Stem cells that have been destroyed by treatment are replaced using BMT and PBSCT.

BMT and PBSCT are most commonly used in the treatment of leukemia and lymphoma. BMT and PBSCT are often used to treat leukemia that is in remission (phase during which the signs and symptoms of cancer have disappeared) and cancers that are not responding to other treatment or have recurred.

In one embodiment of the invention, targeted thermotherapy is administered prior to, during, or after bone marrow or stem cell transplantation, or any combination thereof.

Targeted thermotherapy can also be administered to transplanted bone marrow or stem cells excorporeally, prior to transplantation.

4.7. Targeted Thermotherapy in Combination with Photodynamic Therapy

New techniques have been developed using ceramic-based nanoparticles as drug carriers for photodynamic therapy. Photodynamic therapy is based on light-sensitive molecules, photosensitizers (“PS's”), that tend to concentrate in tumor tissues. When irradiated with light of an appropriate wavelength, PS's absorb light and become excited, transferring their energy to nearby molecular oxygen to form reactive oxygen species (ROS's), which in turn oxidize and damage vital components of nearby tumor cells. Magnetic nanoparticles tagged with antibodies can be coated with photosensitive drugs.

Unfortunately, most PS's are hydrophobic and difficult to prepare in an injectable form. To overcome this problem, PS's are packed in lipids and other hydrophobic delivery vehicles. However, these vehicles have disadvantages (e.g., poor loading, side effects), and all of them tend to cause phototoxic side effects due to drug accumulation in skin and eye tissue. Ceramic-based nanoparticles that are capable of selectively delivering PS's to tumor cells and damaging them can be easily prepared to various specifications, are quite stable, and protect molecules against denaturation caused by extremes in pH or temperature. Such nanoparticles are also biocompatible, and their surfaces can be modified to attach antibodies or other ligands for use in targeting the nanoparticles to specific tissues. Even without such modifications, they are selectively taken up by tumors because the leaky vasculature of tumors causes increased uptake of macromolecules. Silica-based nanoparticles are synthesized and doped with the drug 2-devinyl-2-(1-hexyloxyethyl) pyropheophorbide (HPPH). When activated with a 650-nm laser, the nanoparticles cause significant cell death (i.e., cytolysis).

In one embodiment of the invention, silica-based or other optically activated nanoparticles with a magnetic core are produced. The bioprobes comprising these nanoparticles also comprise a drug. These bioprobes are then irradiated with light to activate the drug, and they are irradiated later with the AMF of the targeted thermotherapy system to further destroy the target via heat. The bioprobes may also be irradiated with light and with AMF simultaneously.

In another embodiment of the invention, photodynamic particles and bioprobes are injected separately and activated either simultaneously or separately from one another.

Photodynamic therapy in combination with targeted thermotherapy may be used alone or in combination with chemotherapy, surgery or both.

4.8. Multiple Combined Therapies

The therapies and combined therapies as disclosed in sections 4.1 to 4.7 hereinabove can be further combined in any combination as deemed suitable for the patient. There may be a disease which can be treated with two (dual therapy) or more therapies. The targeted thermotherapy using nano-sized particles in combination with another therapy may treat two or more diseases.

5. Targeted Thermotherapy and Medical Imaging (MRI PET, SPECT, Bioimpedance)

Small paramagnetic or superparamagnetic particles of ferrite (iron oxide Fe₃O₄ or Fe₂O₃) can be used as paramagnetic contrast medium in magnetic resonance imaging (MRI). These agents exhibit strong T1 relaxation properties, and due to susceptibility differences to their surroundings, they also produce a strongly varying local magnetic field that enhances T2 relaxation to darken the contrast media-containing structures. Very small particles of less than 300 nanometers also remain intravascular for a prolonged period of time. The agents are also referred to as SPIO's (“small particle iron oxides” or “superparamagnetic iron oxides”) and USPIO's (“ultrasmall particle iron oxides” or “ultrasmall superparamagnetic iron oxides”). In one embodiment of the present invention, targeted thermotherapy and MRI are combined. MRI contrast isotopes that target vulnerable plaques, such as Gadolinium-labeled antifibrin nanoparticles, are used. Once these nanoparticles are uptaken by the plaque, AMF is used for destroying the plaque.

Positron emission tomography (PET) is a technique for measuring the concentrations of positron-emitting radioisotopes within the tissue of living patients. A wide range of compounds can be used with PET. These positron-emitting radionuclides have short half-lives and high radiation energies. The primary positron-emitting radionuclides used in PET include Carbon-11, Nitrogen-13, Oxygen-15, and Fluorine-18, with half-lives of 20 min, 10 min, 2 min, and 110 min, respectively. These compounds are commonly known in PET as tracer compounds.

Single photon emission computed tomography (SPECT) involves the detection of gamma rays emitted singly from radioactive atoms, called radionuclides, such as Technetium-99 m and Thallium-201. A radiopharmaceutical is a protein or an organic molecule that has a radionuclide attached to it. The proteins and organic molecules are selected based on their use or absorption properties within the human body. SPECT is used routinely to help diagnose and stage cancer, stroke, liver disease, lung disease and a host of other physiological (functional) abnormalities.

Radioimmunological imaging radionuclides, such as Molybdenum-99, Technetium-99 m, Chromium-51, Copper-64, Dysprosium-165, Ytterbium-169, Indium-111, Iodine-125, Iodine-131, Iridium-192, Iron-59, Phosphorus-32, Potassium-42, Rhodium 186, Rhenium-188, Samarium-153, Selenium-75, Sodium-24, Strontium-89, Xenon-133, Xenon-127, Yttrium-90 or others, are bound to antibodies (sometimes referred to as labeling, tracing or tagging) that will bind to a specific antigenic target. In one embodiment of the present invention, radioimmunological imaging is combined with targeted thermotherapy by attaching the radionuclides directly to the bioprobes. In such a configuration, the uptake process of the bioprobes can be directly imaged.

Bioimpedance is a measure of how well the body impedes electric current flow. Fat has high resistivity, blood lower resistivity. Impedance is measured by applying a small electric current, for example, using two electrodes, and measuring the resulting small voltage with another pair of electrodes. The lower the voltage is, the lower the tissue impedance will be for a given current. Tissue consists of cells and membranes; membranes are thin but have a high resistivity and electrically behave as small capacitors. At high frequencies, the result becomes independent of the capacities of the cell membranes. At low frequencies, however, the membranes impede current flow, and the results are dependent on liquids outside the cells.

In one embodiment of the present invention, one or more of these imaging techniques is used to image the uptake of the bioprobes prior to, during, or after targeted therapy administration.

The methods of the present invention may be used to treat a variety of indications which include, but are not limited to, cancer of any type, such as bone marrow, lung, vascular, neuro, colon, ovarian, stomach, rectal, breast, gastric, pancreatic and prostate cancer, melanoma, epitheleoid sarcomas, AIDS, autoimmune conditions, adverse angiogenesis, amyloidosis, cardiovascular plaque, vascular plaque, calcified plaque, vulnerable plaque, restenosis, vascular conditions, tuberculosis, obesity, malaria, and illnesses due to viruses, such as HIV.

While the above description of the invention has been presented in terms of a human subject (patient), it is appreciated that the invention may also be applicable to treating other subjects, such as mammals, organ donors, cadavers and the like.

As noted above, the present invention is applicable to targeted thermotherapeutic compositions, systems and methods for treating diseased tissue, pathogens, or other undesirable matter that involve the administration of energy susceptive materials, that are attached to a target-specific ligand, to a patient's body, body part, tissue, or body fluid, and the administration of an energy source to the energy susceptive materials. The targeted methods can be used in combination with at least one other treatment method. The present invention should not be considered limited to the particular embodiments described above, but rather should be understood to cover all aspects of the invention as fairly set out in the appended claims. Various modifications, equivalent processes, as well as numerous structures to which the present invention may be applicable will be readily apparent to those skilled in the art to which the present invention is directed upon review of the present specification. The claims are intended to cover such modifications and devices.

1. A targeted thermotherapy system for treating disease material in a patient, the system comprising:
a) a bioprobe or a bioprobe system comprising a susceptor;
b) an alternating magnetic field (AMF) inducing inductor that produces an AMF to energize the susceptor; and
c) a generator coupled to the inductor to provide power to the AMF inducing inductor.

2. The system according to claim 1, wherein the inductor comprises an AMF inducing inductor having a core defining at least part of a magnetic circuit, the core having two poles, the two poles of the core defining a gap therebetween, and a magnetic field passing between the two poles.

3. The system according to claim 1, wherein the inductor comprises a coil that surrounds a patient and has at least one turn.

4. The system according to claim 1, wherein the inductor comprises a coil placed dorsal or anterior to a patient.

5. The system according to claim 1, wherein the inductor comprises at least one gradient coil of a nuclear magnetic resonance imaging (MRI) system.

6. The system according to claim 5, wherein the inductor comprises a plurality of gradient coils of a MRI system switched sequentially to generate the AMF.

7. The system according to claim 5, wherein the inductor comprises a plurality of gradient coils of a MRI system, the plurality of gradient coils being switched sequentially to generate a rotating AMF.

8. The system according to claim 1, further comprising at least one pair of pulse modulators, wherein the at least one pair of pulse modulators is coupled to the inductor in opposite polarity to produce an alternating current in the inductor.

9. The system according to claim 1, wherein the magnetic inductor comprises:
a. a circular rotor; and
b. at least two magnets attached to or mounted on the circular rotor to create a magnetic flux,
wherein there is a gap between the magnets, and wherein the circular rotor rotates around a target located within the gap.

10. The system according to claim 9, wherein the circular rotor builds a return path for the magnetic flux of the magnets.

11. The system according to claim 9, wherein the circular rotor is fabricated from a low magnetic reluctance material.

12. The system according to claim 1, wherein the bioprobe comprises one or more ligands.

13. The system according to claim 1, wherein the bioprobe comprises one or more antibodies.

14. The system according to claim 13, wherein the antibody comprises AC10, HeFi1, derivatives of AC10 and HeFi1, 19D9D6 Monoclonal Antibody, MV833, HuMV833, Anti-cytokeratin AE1/3, anti-CAM5.2, M170, chimeric M170, Votumumab, Mab 88BV59, ABX-EGF, HuMax-EGFr, h-R3, 4B5-H, ABX-MA1, MDX-010, Mab-1A7, ACA-125, R1549, Pemtumomab, MuHMFg1, HuHMFg1, Mab-B42.13, Ov, VB2-011, H-11 ScFv, Novo Mab-G2ScFv, Bevacizumab, rhuMAb-VEGF, SGN-15, cBR96, Pertuzumab, rhuMAb 2C4, Mab AR20.5, R1550, huHMFG1, ING-1, huLM609, Mab-MEDI-522, huLM609, or a combination thereof.

15. The system according to claim 1, wherein the bioprobe comprises antifibrin.

16. The system according to claim 1, wherein the bioprobe susceptor comprises iron oxide.

17. The system according to claim 1, further comprising one or more bioprobes.

18. The system according to claim 17, wherein the bioprobes are distinct from one another.

19. A therapeutic method for treating the body, body part, tissue, cell, or body fluid of a subject, comprising:
a. administering targeted thermotherapy to a target by supplying a bioprobe to the target and exposing the bioprobe to an alternating magnetic field (AMF), and
b. administering at least one other therapy to the target,
wherein the at least one other therapy is administered prior to, during, after the targeted thermotherapy administration, or a combination thereof.

20. The therapeutic method according to claim 19, wherein administering the at least one other therapy comprises administering a sensitizing drug that induces the coagulation in the vasculature of a tumor.

21. A therapeutic method according to claim 20, wherein the sensitizing drug comprises monophosphoryl lipid A (MPL), monocyte chemoattractant protein-1 (MCP-1), platelet-derived growth factor-BB (PDGF-BB), C-reactive protein (CRP), tumor necrosis factor-α (TNF-α) or an inducer of TNF-α, a Rac1 antagonist, DMXAA, CM101 or thalidomide, muramyl dipeptide (MDP), threonyl-MDP or MTPPE, anti-angiogenic agent, vasculostatin, canstatin or maspin, VEGF inhibitor, anti-VEGF blocking antibody, VEGF receptor construct (sVEGF-R), tyrosine kinase inhibitor, antisense VEGF construct, anti-VEGF RNA aptamer, anti-VEGF ribozyme, antibody that binds to the cell surface activating antigen CD40, sCD40-Ligand (sCD153), combretastatin A-1, A-2, A-3, A-4, A-5, A-6, B-1, B-2, B-3, B-4, D-1 or D-2, thalidomide, or a combination thereof.

22. A therapeutic method according to claim 20, wherein the sensitising drug comprises an antibody, antigen-binding region, monoclonal, recombinant, human, part-human, humanized or chimeric antibody or antigen-binding region, scFv, Fv, Fab′, Fab, diabody, linear antibody or F(ab′)₂, ligand, growth factor or receptor, VEGF receptor, FGF receptor, TGF-β receptor, TIE, VCAM-1, ICAM-1, P-selectin, E-selectin, PSMA, pleiotropin, endosialin or endoglin, fibronectin, scatter factor/hepatocyte growth factor (HGF), platelet factor 4 (PF4), PDGF, or a combination thereof.

23. A therapeutic method according to claim 19, wherein the at least one other therapy comprises hyperthermia.

24. A therapeutic method according to claim 23, wherein the hyperthermia comprises RF eddy current, light, direct RF or microwave radiation, alternating or direct currents, induction of thermal seeds, thermal baths of hot or warm water, oils or other solutions, induction of non-targeted particles, ionising radiation, or any combination thereof.

25. A therapeutic method according to claim 19, wherein the at least one other therapy comprises monoclonal antibody therapy.

26. A therapeutic method according to claim 19, wherein the at least one other therapy comprises radiation therapy.

27. A therapeutic method according to claim 26, wherein the radiation therapy comprises radio immunotherapy, and wherein the radio immunotherapy comprises use of a radionuclide comprising Molybdenum-99, Technetium-99 m, Chromium-51, Copper-64, Dysprosium-165, Ytterbium-169, Indium-111, Iodine-125, Iodine-131, Iridium-192, Iron-59, Phosphorus-32, Potassium-42, Rhodium 186, Rhenium-188, Samarium-153, Selenium-75, Sodium-24, Strontium-89, Xenon-133, Xenon-127, and Yttrium-90 or a combination thereof.

28. The therapeutic method according to claim 26, wherein the radiation therapy is radio immunotherapy, and wherein the radio immunotherapy comprises use of a radionuclide associated with a monoclonal antibody or a bioprobe of the targeted thermotherapy system.

29. The therapeutic method according to claim 19, wherein the at least one other therapy comprises chemotherapy.

30. The therapeutic method according to claim 29, wherein the chemotherapy comprises administering a drug or agent, wherein the drug or agent comprises an S phase-dependent antimetabolics, capercitabine, cytarabine, doxorubicin, fludarabine, floxuridine, fluorouracil, gemcitabine, hydroxyurea, mercaptopurine, methotrexate, prednisone, procarbazine, thioguanine, M phase-dependent vinca alkaloids, vinblastine, vincristine, vinorelbine, podophyllotoxins, etoposide, teniposide, taxanes, doxetaxel, paxlitaxel, G₂ pase-dependent, bleomycin, irinotecan, mitoxantrone, topotecan, G₁ pase-dependent, asparaginase, corticosteroids, alkylating agents, nitrogen mustards, mechlorethamine, mustargen, cyclophosphamide, ifosfamide (Ifex), and chlorambucil, leukeran, nitrosoureas, platinum agents, cisplatin, platinol, carboplatin, paraplatin, antimetabolites, natural therapeutic products, antitumor antibiotics, bleomycin, anthracyclines, epipodophyllotoxins, vinca alkaloids, taxanes, camptothecin, or a combination thereof.

31. The therapeutic method according to claim 29, wherein the chemotherapy comprises administering a drug or agent, wherein the drug or agent is associated with a monoclonal antibody or to the bioprobe.

32. The therapeutic method according to claim 29, wherein the chemotherapy comprises administering a drug or agent associated with the bioprobe, wherein the drug or agent is activated during AMF exposure by being released from the bioprobe.

33. The therapeutic method according to claim 29, wherein the chemotherapy comprises administering a drug or agent, wherein the drug or agent is destroyed when exposed to the AMF.

34. The therapeutic method according to claim 29, wherein the bioprobe comprises a coating, and wherein the chemotherapy comprises administering a drug or agent that is intercalated into the coating of the bioprobe.

35. The therapeutic method according to claim 19, wherein the at least one other therapy comprises pharmaceutical therapy.

36. The therapeutic method according to claim 35, wherein the pharmaceutical therapy comprises one or more vasopermeation enhancement agents.

37. The therapeutic method according to claim 19, wherein the at least one other therapy comprises surgery, minimally invasive surgery, or an interventional technique.

38. The therapeutic method according to claim 37, further comprising surgically preparing an organ to be lifted outside the body while the organ continues to being anatomically and physiologically attached to the body, and extracorporeally irradiating the organ with the AMF.

39. The therapeutic method according to claim 19, wherein the at least one other therapy comprises bone marrow or stem cell transplantation.

40. The therapeutic method according to claim 19, wherein the at least one other therapy comprises administering Bevacizumab, rhuMAb-VEGF, BMS-275291, Celecoxib, EMD121974, rhEndostatin, cetuximab, Interferon-α, LY317615, AE-941, PTK787, SU6668, SU11248, Thalidomide, ZD1839, ZD6474, or a combination thereof.

41. A therapeutic method according to claim 19, wherein the at least one other therapy comprises photodynamic therapy.

42. The therapeutic method according to claim 41, wherein the photodynamic therapy comprises administering at least one photodynamic particle which comprises a silica-based or other optically activated nanoparticle with a magnetic core, and a drug, wherein the at least one photodynamic particle is irradiated with light to activate the drug.

43. The therapeutic method according to claim 42 wherein the at least one photodynamic particle and bioprobes are injected into the patient separately and activated simultaneously.

44. The therapeutic method according to claim 42, wherein the at least one photodynamic particle and bioprobes are injected into the patient separately and activated separately.

45. A therapeutic method, comprising:
a. administering targeted thermotherapy to a body, body part, or tissue of a subject containing a tumor, by supplying a bioprobe to the body, body part or tissue and exposing the bioprobe to an alternating magnetic field (AMF), and
b. destroying or inhibiting the vascularity of the body, body part or tissue in response to exposure to the AMF.

46. The therapeutic method according to claim 45, further comprising administering at least one other therapy to the body, body part or tissue.

47. The therapeutic method according to claim 46, further comprising administering an agent, the agent comprising a sensitizing drug that induces the coagulation of the vasculature in a tumor.

48. The therapeutic method according to claim 47, wherein the sensitizing drug comprises monophosphoryl lipid A (MPL), monocyte chemoattractant protein-1 (MCP-1), platelet-derived growth factor-BB (PDGF-BB), C-reactive protein (CRP), tumor necrosis factor-α (TNF-α) or an inducer of TNF-α, a Rac1 antagonist, DMXAA, CM101 or thalidomide, muramyl dipeptide (MDP), threonyl-MDP or MTPPE, anti-angiogenic agent, vasculostatin, canstatin or maspin, VEGF inhibitor, anti-VEGF blocking antibody, VEGF receptor construct (sVEGF-R), tyrosine kinase inhibitor, antisense VEGF construct, anti-VEGF RNA aptamer, anti-VEGF ribozyme, antibody that binds to the cell surface activating antigen CD40, sCD40-Ligand (sCD153), combretastatin A-1, A-2, A-3, A-4, A-5, A-6, B-1, B-2, B-3, B-4, D-1 or D-2, thalidomide, or a combination thereof.

49. The therapeutic method according to claim 47, wherein the sensitising drug comprises an antibody, antigen-binding region, monoclonal, recombinant, human, part-human, humanized or chimeric antibody or antigen-binding region, scFv, Fv, Fab′, Fab, diabody, linear antibody or F(ab′)₂, ligand, growth factor or receptor, VEGF receptor, FGF receptor, TGF-β receptor, TIE, VCAM-1, ICAM-1, P-selectin, E-selectin, PSMA, pleiotropin, endosialin or endoglin, fibronectin, scatter factor/hepatocyte growth factor (HGF), platelet factor 4 (PF4), PDGF, or a combination thereof.

50. The therapeutic method according to claim 46, wherein the at least one other therapy comprises hyperthermia.

51. The therapeutic method according to claim 50, wherein the hyperthermia comprises RF eddy current, light, direct RF or microwave radiation, alternating or direct currents, induction of thermal seeds, thermal baths of hot or warm water, oils or other solutions, induction of non-targeted particles, ionising radiation, or any combination thereof.

52. The therapeutic method according to claim 46, wherein the at least one other therapy comprises monoclonal antibody therapy.

53. The therapeutic method according to claim 52, wherein the monoclonal antibody therapy comprises administering an antibody, and wherein the antibody comprises AC10, HeFi1, derivatives of AC10 and HeFi1, 19D9D6 Monoclonal Antibody, MV833, HuMV833, Anti-cytokeratin AE1/3, anti-CAM5.2, M170, chimeric M170, Votumumab, Mab 88BV59, ABX-EGF, HuMax-EGFr, h-R3, 4B5-H, ABX-MA1, MDX-010, Mab-1A7, ACA-125, R1549, Pemtumomab, MuHMFg1, HuHMFg1, Mab-B42.13, Ov, VB2-011, H-11 ScFv, Novo Mab-G2ScFv, Bevacizumab, rhuMAb-VEGF, SGN-15, cBR96, Pertuzumab, rhuMAb 2C4, Mab AR20.5, R1550, huHMFG1, ING-1, huLM609, Mab-MEDI-522, huLM609, or a combination thereof.

54. The therapeutic method according to claim 46, wherein the at least one other therapy comprises radiation therapy.

55. The therapeutic method according to claim 54, wherein the radiation therapy comprises radio immunotherapy, and wherein the radio immunotherapy comprises administering a radionuclide which comprises of Molybdenum-99, Technetium-99 m, Chromium-51, Copper-64, Dysprosium-165, Ytterbium-169, Indium-111, Iodine-125, Iodine-131, Iridium-192, Iron-59, Phosphorus-32, Potassium-42, Rhodium 186, Rhenium-188, Samarium-153, Selenium-75, Sodium-24, Strontium-89, Xenon-133, Xenon-127, and Yttrium-90, or a combination hereof.

56. The therapeutic method according to claim 54 wherein the radiation therapy comprises radio immunotherapy, and wherein the radio immunotherapy comprises administering a radionuclide bound to a monoclonal antibody or the bioprobe.

57. The therapeutic method according to claim 46, wherein the at least one other therapy comprises chemotherapy.

58. The therapeutic method according to claim 57, wherein the chemotherapy comprises administering a drug or agent, and wherein the drug or agent comprises an S phase-dependent antimetabolics, capercitabine, cytarabine, doxorubicin, fludarabine, floxuridine, fluorouracil, gemcitabine, hydroxyurea, mercaptopurine, methotrexate, prednisone, procarbazine, thioguanine, M phase-dependent vinca alkaloids, vinblastine, vincristine, vinorelbine, podophyllotoxins, etoposide, teniposide, taxanes, doxetaxel, paxlitaxel, G₂ pase-dependent, bleomycin, irinotecan, mitoxantrone, topotecan, G₁ pase-dependent, asparaginase, corticosteroids, alkylating agents, nitrogen mustards, mechlorethamine, mustargen, cyclophosphamide, ifosfamide (Ifex), and chlorambucil, leukeran, nitrosoureas, platinum agents, cisplatin, platinol, carboplatin, paraplatin, antimetabolites, natural therapeutic products, antitumor antibiotics, bleomycin, anthracyclines, epipodophyllotoxins, vinca alkaloids, taxanes, camptothecin, or a combination thereof.

59. The therapeutic method according to claim 57, wherein the chemotherapy comprises administering a drug or agent, wherein the drug or agent is associated with a monoclonal antibody or the bioprobe.

60. The therapeutic method according to claim 57, wherein the chemotherapy comprises administering a drug or agent, wherein the drug or agent is activated during the AMF exposure by being released from the bioprobe.

61. The therapeutic method according to claim 57, wherein the chemotherapy comprises administering a drug or agent, wherein the drug or agent is destroyed upon exposure to the AMF.

62. The therapeutic method according to claim 57, wherein the chemotherapy comprises administering a drug or agent, wherein the drug or agent is intercalated into a coating of the bioprobe.

63. The therapeutic method according to claim 46, wherein the at least one other therapy comprises pharmaceutical therapy.

64. The therapeutic method according to claim 63, wherein the pharmaceutical therapy comprises administering one or more vasopermeation enhancement agents.

65. The therapeutic method according to claim 46, wherein the at least one other therapy comprises surgery, minimally invasive surgery, or an interventional technique.

66. The therapeutic method according to claim 65, further comprising surgically preparing an organ to be lifted outside the body, while the organ continues to being anatomically and physiological attached to the body, and extracorporeally exposing the organ to the AMF.

67. The therapeutic method according to claim 46, wherein at least one other therapy comprises bone marrow or stem cell transplantation.

68. The therapeutic method according to claim 46, wherein the at least one other therapy comprises administering Bevacizumab, BMS-275291, Celecoxib, EMD121974, rhEndostatin, cetuximab, Interferon-α, LY317615, AE-941, PTK787, SU6668, SU11248, Thalidomide, ZD1839, ZD6474, or a combination thereof.

69. The therapeutic method according to claim 46, wherein the at least one other therapy comprises photodynamic therapy.

70. The therapeutic method according to claim 69, wherein the photodynamic therapy comprises administering at least one photodynamic particle which comprises a silica-based or other optically activated nanoparticle with a magnetic core, and a drug, and irradiating the at least one photodynamic particle with light to activate the drug.

71. The therapeutic method according to claim 70, further comprising introducing the at least one photodynamic particle and the bioprobes to the body, body part or tissue separately and activating the at least one photodynamic particle and bioprobe either simultaneously or separately from one another.

72. A therapeutic method for treating the body, body part, tissue, cell, or body fluid of a subject, comprising:
a) medically imaging the body, body part, tissue, cell or body fluid; and
b) administering targeted thermotherapy by introducing a bioprobe to the body, body part, tissue, cell or body fluid of the subject and exposing the bioprobe to an alternating magnetic field (AMF),
wherein the administering the targeted thermotherapy occurs prior to, during, or after the medical imaging, or a combination thereof.

73. The therapeutic method according to claim 72, wherein medically imaging the body, body part, tissue, cell or body fluid comprises use of magnetic resonance imaging, x-ray imaging, positron emission tomography, single photon emission computed tomography, bioimpedance measurements, radioimmunological imaging, or a combination thereof.

74. The therapeutic method according to claim 73, wherein the radioimmunological imaging comprises administering to the patient at least one radionuclide, and wherein the radionuclide comprises Molybdenum-99, Technetium-99 m, Chromium-51, Copper-64, Dysprosium-165, Ytterbium-169, Indium-111, Iodine-125, Iodine-131, Iridium-192, Iron-59, Phosphorus-32, Potassium-42, Rhodium 186, Rhenium-188, Samarium-153, Selenium-75, Sodium-24, Strontium-89, Xenon-133, Xenon-127, or Yttrium-90 or a combination of these radionuclides.

75. The therapeutic method according to claim 74, wherein the medical imaging comprises administering to the patient at least one radionuclide, the at least one radionuclide being attached to the bioprobe.

76. The therapeutic method according to claim 73, wherein the medical imaging comprises magnetic resonance imaging (MRI), and the bioprobe comprises antifibrin and is gadolinium-labeled.

77. The therapeutic method according to claim 72, further comprising administering at least one other therapy, wherein the at least one other therapy comprises hyperthermia, direct antibody therapy, radiation therapy, chemotherapy or pharmaceutical therapy, photodynamic therapy, surgical therapy, interventional therapy, bone marrow or stem cell transplantation, or a combination thereof.

78. A magnetic material composition, comprising:
a. a particle having magnetic properties and forming a single magnetic domain;
b. a biocompatible coating material for the particle; and
c. a ligand selective to at least one disease material marker associated with disease material, the ligand being i) bound to an uncoated portion of the particle, ii) bound to a coated portion of the particle, iii) bound to the particle and partially covered by the coating or iv) intercalated into the coating.

79. The magnetic particle composition of claim 78, wherein the biocompatible coating material is biodegradable.

80. The magnetic particle composition of claim 78, wherein the particle has a size of no more than about 250 nm in at least one dimension.

81. The magnetic material composition of claim 78, wherein the particle, the coating and the ligand are suspended in a biologically compatible fluid.

82. The magnetic material composition of claim 78, wherein the magnetic particle is ferromagnetic, antiferromagnetic, ferrimagnetic, antiferrimagnetic or superparamagnetic.

83. A magnetic material composition of claim 78, wherein the magnetic particle comprises an iron oxide prepared via a synthetic process, natural process, or a combination thereof.

84. A magnetic material composition of claim 83, wherein the iron oxide is prepared by biologically induced mineralization, boundary organized biomineralization, or a combination thereof.

85. A magnetic material composition of claim 84, wherein the boundary organized biomineralization process occurs in one species of magnetotactic bacteria.

86. A magnetic material composition of claim 78, wherein the magnetic particle has a Curie temperature in the range of from about 40° C. to about 150° C.

87. A magnetic material composition of claim 78, wherein the magnetic particle is formed of a biocompatible material, and wherein the surface of the magnetic particle forms the biocompatible coating.

88. A magnetic material composition of claim 78, wherein the biocompatible coating material is an organic material, an inorganic material, or a combination thereof.

89. A magnetic material composition of claim 88, wherein the organic material is a synthetic material, a biological material, or a combination thereof.

90. A magnetic material composition of claim 89, wherein the synthetic material is a polymer, a copolymer, or a combination thereof.

91. A magnetic material composition of claim 89, wherein the synthetic material comprises at least a polymer, a copolymer, or a polymer blend formed from a polymer based on at least one of acrylates, siloxanes, styrenes, acetates, alkylene glycols, alkylenes, alkylene oxides, parylene, lactic acid, and glycolic acid.

92. A magnetic material composition of claim 89, wherein the synthetic material comprises a hydrogel polymer, a histidine-containing polymer, a surfactant, or a combination thereof.

93. A magnetic material composition of claim 89, wherein the biological material comprises at least one of a polysaccharide, a polyaminoacid, a protein, a lipid, a glycerol, a fatty acid, and a combination thereof.

94. A magnetic material composition of claim 93, wherein the polysaccharide includes a heparin, heparin sulfate, chondroitin sulfate, chitin, chitosan, cellulose, dextran, alginate, starch, saccharide, carbohydrate, glycosaminoglycan, or a combination thereof.

95. A magnetic material composition of claim 93, wherein the protein includes an extracellular matrix protein, proteoglycan, glycoprotein, albumin, peptide, gelatin, or a combination thereof.

96. A magnetic material composition of claim 88, wherein the inorganic material includes a metal, a metal alloy, a ceramic, an oxide of a Group IV element, or a combination thereof.

97. A magnetic material composition of claim 96, wherein the ceramic includes hydroxyapatite, silicon carbide, carboxylate, sulfonate, phosphate, ferrite, phosphonate, or a combination thereof.

98. A magnetic material composition of claim 89, wherein the biological material is a transfection agent to enhance uptake by cancer cells.

99. A magnetic material composition of claim 98, wherein the transfection agent includes a vector, a prion, a polyaminoacid, a cationic liposome, an amphiphile, a non-liposomal lipid, or a combination thereof.

100. A magnetic material composition of claim 99, wherein the vector includes a plasmid, a virus, a phage, a viron, a viral coat, or a combination thereof.

101. A therapeutic method according to claim 19, wherein the targeted thermotherapy is administered using a targeted thermotherapy system that comprises a plurality of different bioprobes or bioprobe systems, a magnetic generator, and an inductor.

102. A therapeutic method according to claim 19, wherein the method is utilized for the treatment of a cancer, AIDS, adverse angiogenesis, cardiovascular plaque, vascular plaque, calcified plaque, vulnerable plaque, restenosis, amyloidosis, tuberculosis, obesity, malaria, and illnesses due to viruses.

103. A magnetic material composition, comprising:
a. a bioprobe, the bioprobe comprising a particle having magnetic properties associated with a first therapy, and a ligand selective to at least one disease material marker associated with a disease material; the ligand being associated with the particle; and
b. an agent associated with a second therapy, the agent being associated with the bioprobe.

104. The composition of claim 103, wherein the agent comprises a radiotherapeutic agent,

105. The composition of claim 104, wherein the radiotherapeutic agent comprises a radionuclide.

106. The composition of claim 103, wherein the agent comprises a chemotherapeutic agent.

107. The composition of claim 103, wherein the agent comprises a pharmaceutical agent.

108. The composition of claim 103, wherein the agent comprises a photodynamic agent.

109. The composition of claim 103, wherein the bioprobe further comprises a coating.

110. The composition of claim 103, wherein the bioprobe forms a single domain.
